(19) United States
(12) Patent Application Publication
(10) Pub. No.: US 2023/0049079 A1
(43) Pub. Date: Feb. 16, 2023

(54) DELIVERY SYSTEMS AND DELIVERY ASSEMBLIES FOR PROSTHETIC HEART VALVES, METHODS OF MAKING AND USING THE SAME

(71) Applicant: Edwards Lifesciences Corporation, Irvine, CA (US)

(72) Inventor: Erik Bulman, Lake Forest, CA (US)

(21) Appl. No.: 17/978,927

(22) Filed: Nov. 1, 2022

Related U.S. Application Data
(63) Continuation of application No. PCT/US2021/030427, filed on May 3, 2021.
(60) Provisional application No. 63/020,335, filed on May 5, 2020.

(51) Int. Cl. A61F 2/24; A61M 25/01; A61M 25/00

(52) U.S. Cl. CPC A61F 2/2436; A61M 25/0147; A61M 25/005; A61M 25/0045

(57) ABSTRACT

Disclosed herein are delivery systems and delivery assemblies comprising a co-extruded elongated polymeric sleeve comprising an inner polymer layer and an outer polymer layer, which form two lumens having a different diameter, wherein the inner polymer layer comprises a fluoropolymer; and wherein the outer polymer layer comprises a polyamide, or a polyether block amide, or a combination thereof. Also disclosed are methods of making and using the same.

### CROSS-REFERENCE TO RELATED APPLICATIONS

This application is a continuation of International Application No. PCT/US2021/030427, filed May 3, 2021, which claims the benefit of U.S. Provisional Application No. 63/020,335, filed May 5, 2020, the content of which is incorporated herein in its entirety.

### FIELD

The present application relates to delivery systems, delivery assembles for prosthetic heart valves, methods of making, and using the same.

### BACKGROUND

The present disclosure relates to systems used to deliver a prosthetic valve to a heart. More specifically, the present disclosure is directed to an improved steerable delivery system for delivery of a prosthetic valve to a human heart.

Catheters are known in the art and have been commonly used to reach locations inside the body that are not readily accessible by surgery or where access without surgery is desirable. The usefulness of catheters is largely limited by the ability of the catheter to successfully navigate through small vessels and around tight bends, such as around the aortic arch.

Over the years, a variety of steerable catheters have been proposed for facilitating navigation through difficult vasculature. For example, some known devices employ a series of connected segments, each comprising a shape that allows the catheter to form a bent configuration adaptable to fit the particular need. The use of many connected segments, however, is complicated and costly.

Also known in the art is a device wherein portions have been removed from a hollow stylet wire, thus allowing the hollow wire to bend in areas where portions have been removed. However, known devices of this type are used as stylets and have not been adapted for use in a steerable catheter.

Also known in the art is a device wherein spring bands are employed into a steerable catheter, wherein one spring band has a natural curvature opposite that of the direction of the bending of the device, thus providing stability to the device. However, these bands add unnecessary complexity to the device and are therefore undesirable for many uses.

Although a variety of bendable and steerable devices have been proposed over the years, each of the existing devices has shortcomings that limit its effectiveness. Accordingly, an urgent need exists for an improved steerable delivery system to facilitate advancement of an implant and/or therapy device through a patient's vasculature to a treatment site. It is desirable that such a system overcomes the shortcomings associated with existing devices. It is also desirable that such a system be versatile, reliable, and easy to use. The present disclosure addresses this need.

### SUMMARY

The aspects of the present disclosure relate to a delivery system for deploying a prosthetic valve comprising: an elongated polymeric sleeve having a proximal end and a distal end and comprising an inner polymer layer and an outer polymer layer; wherein the inner polymer layer forms: a first tubular sleeve having an inner surface defining a first lumen having a first diameter, an outer surface, and a first longitudinal axis; and a second tubular sleeve that is permanently adjacent to the first tubular sleeve, wherein the second tubular sleeve has an inner surface defining a second lumen having a second diameter, and an outer surface that is an extension of the outer surface of the first tubular sleeve and wherein the second tubular sleeve has a second longitudinal axis; wherein the second diameter is substantially smaller than the first diameter; and wherein the second longitudinal axis is offset of the first longitudinal axis and is substantially parallel to the first longitudinal axis along a length of the elongated polymeric sleeve; and wherein the outer surface of the first tubular sleeve and the second tubular sleeve defines an outer surface of the inner polymer layer; and wherein the inner surface of the first tubular sleeve defines an inner surface of the elongated polymeric sleeve, wherein the outer polymer layer extends circumferentially around the inner polymer layer, wherein the outer polymer layer has an inner surface and an outer surface, wherein the inner surface of the outer polymer layer is adjacent and bonded to the outer surface of the inner polymer layer; wherein the inner polymer layer comprises a fluoropolymer; and wherein the outer polymer layer comprises a polyamide, or a polyether block amide, or a combination thereof.

In such exemplary aspects, the inner polymer layer and the outer polymer layer of the elongated polymeric sleeve are co-extruded.

Also disclosed herein is a delivery system for deploying a prosthetic valve comprising: a first tubular sleeve having an inner polymer layer and an outer polymer layer, wherein the inner polymer layer defines an inner surface of the first tubular sleeve that forms a first lumen having a first diameter, and wherein the outer polymer layer is disposed on the inner polymer layer and defines an outer surface of the first tubular sleeve, and wherein the first tubular sleeve has a first longitudinal axis; and optionally, a second tubular sleeve that is substantially aligned with the first tubular sleeve, wherein the second tubular sleeve has an inner polymer layer and an outer polymer layer, wherein the inner polymer layer defines an inner surface of the second tubular sleeve that forms a second lumen having a second diameter, and wherein the outer polymer layer is disposed on the inner polymer layer and defines an outer surface of the second tubular sleeve, and wherein the second tubular sleeve has a second longitudinal axis; wherein the second diameter is substantially smaller than the first diameter; and wherein the second longitudinal axis is offset of the first longitudinal axis and is substantially parallel to the first longitudinal axis along a length of the elongated polymeric sleeve, wherein the inner polymer layer of the first tubular sleeve comprises a fluoropolymer; wherein the outer polymer layer of the first tubular sleeve comprises a polyamide, or a polyether block amide, or a combination thereof; and wherein the inner polymer layer and the outer polymer layers are coextruded.

In still further aspects, the inner polymer layer of any of the sleeves disclosed herein is substantially free of delamination. In yet still, further aspects, the fluoropolymer of any one of the inner polymer layers can comprise an ethylene-perfluoroethylenepropene copolymer. While in other aspects, the outer polymer layer can comprise a polyamide comprising nylon 12.

Also disclosed herein are assemblies comprising: a self-expanding prosthetic valve comprising a radially compressible and expandable metallic stent and a flexible valvular structure mounted within the stent; and a delivery apparatus comprising any of the disclosed herein delivery systems.

Also disclosed herein are methods of making of any of the disclosed herein delivery systems. In some aspects, disclosed herein is a method of making a delivery sleeve assembly comprising: forming an elongated polymeric sleeve having a proximal end and a distal end and comprising an inner polymer layer and an outer polymer layer; wherein the inner polymer layer forms: a first tubular sleeve having an inner surface defining a first lumen having a first diameter, an outer surface, and a first longitudinal axis; and a second tubular sleeve that is permanently adjacent to the first tubular sleeve, wherein the second tubular sleeve has an inner surface defining a second lumen having a second diameter, and an outer surface that is an extension of the outer surface of the first tubular sleeve and wherein the second tubular sleeve has a second longitudinal axis; wherein the second diameter is substantially smaller than the first diameter; and wherein the second longitudinal axis is offset of the first longitudinal axis and is substantially parallel to the first longitudinal axis along a length of the elongated polymeric sleeve; and wherein the outer surface of the first tubular sleeve and the second tubular sleeve defines an outer surface of the inner polymer layer; and wherein the inner surface of the first tubular sleeve defines an inner surface of the elongated polymeric sleeve, wherein the outer polymer layer extends circumferentially around the inner polymer layer, wherein the outer polymer layer has an inner surface and an outer surface, wherein the inner surface of the outer polymer layer is adjacent and bonded to the outer surface of the inner polymer layer; wherein the step of forming comprises co-extruding a first polymer to form the inner polymer layer and a second polymer to form the outer polymer layer.

Still further is also disclosed a method of making a delivery sleeve assembly comprising: forming a first tubular sleeve having an inner polymer layer and an outer polymer layer, wherein the inner polymer layer defines an inner surface of the first tubular sleeve that forms a first lumen having a first diameter, and wherein the outer polymer layer is disposed on the inner polymer layer and defines an outer surface of the first tubular sleeve, and wherein the first tubular sleeve has a first longitudinal axis; and optionally, forming a second tubular sleeve that is substantially aligned with the first tubular sleeve, wherein the second tubular sleeve has an inner polymer layer and an outer polymer layer, wherein the inner polymer layer defines an inner surface of the second tubular sleeve that forms a second lumen having a second diameter, and wherein the outer polymer layer is disposed on the inner polymer layer and defines an outer surface of the second tubular sleeve, and wherein the second tubular sleeve has a second longitudinal axis; wherein the second diameter is substantially smaller than the first diameter; and wherein the second longitudinal axis is offset of the first longitudinal axis and is substantially parallel to the first longitudinal axis along a length of the elongated polymeric sleeve, wherein the inner polymer layer of the first tubular sleeve comprises a fluoropolymer; wherein the outer polymer layer of the first tubular sleeve comprises a polyamide, or a polyether block amide, or a combination thereof; and wherein the inner polymer layer and the outer polymer layers are coextruded.

Additional aspects of the disclosure will be set forth, in part, in the detailed description, figures, and claims which follow, and in part will be derived from the detailed description or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

Features and advantages of the present disclosure will become appreciated as the same become better understood with reference to the specification, claims, and appended drawings wherein:

FIG. 1 is a side view of the heart valve delivery system delivering a heart valve to a native valve site according to one aspect of the present disclosure.

FIG. 2 is a cross-sectional view of a handle used in the delivery system.

FIGS. 3A and 3B are perspective and cross-sectional views, respectively, of a first core member, which forms a portion of the handle.

FIGS. 4A and 4B are perspective and a cross-sectional view, respectively, of a partially threaded member, which is disposed around the core member.

FIGS. 5A and 5B are side and cross-sectional views, respectively, of a rotator handle.

FIGS. 6A and 6B are perspective and cross-sectional views, respectively, of a second core member, which forms another portion of the handle.

FIGS. 7A and 7B are perspective and cross-sectional views, respectively, of a hub that is disposed around the second core member.

FIG. 8 is a side view of a guide tube positioned in a handle having a passageway for slidably receiving a pull wire.

FIGS. 9A-9F show an exemplary elongated polymeric sleeve in various aspects: FIG. 9A shows a cross-section view of an exemplary elongated sleeve and a tubing used to manipulate a pull wire as used in the prior art; FIG. 9B shows a cross-section view of an exemplary elongated sleeve according to the aspects of the present disclosure; FIG. 9C shows a perspective view of an exemplary sleeve according to one aspect of the present disclosure; FIG. 9D shows a perspective view of an exemplary sleeve according to a different aspect of the present disclosure;

FIGS. 9E and 9F show additional views of an exemplary sleeve according to additional aspects disclosed herein.

FIG. 10 is a cross-sectional view of a distal portion of a delivery sleeve assembly.

FIG. 11 is a side view of a flex tube that provides a steerable section, wherein the flex tube has been laid flat for purposes of illustration.

FIG. 12 is a cross-sectional view of a portion of a delivery sleeve assembly according to an alternative aspect.

FIG. 13 is a cross-sectional view of a shroud section of the delivery sleeve assembly.

FIGS. 14A and 14B are perspective and cross-sectional views, respectively, of a shroud which forms a portion of the shroud section of FIG. 13.

FIGS. 15A, 15B, and 15C are perspective, cross-sectional, and bottom views, respectively, of a ring that forms a portion of the shroud section of FIG. 13.

FIG. 16 is a cross-sectional view of a balloon catheter configured for use with the heart valve delivery system.

FIGS. 17A and 17B are perspective and cross-sectional views, respectively, of a balloon which forms a portion of the balloon catheter of FIG. 16.

FIGS. 18A and 18B are cross-sectional views of a distal end of the delivery system, wherein FIG. 18A illustrates a first aspect with the prosthetic heart valve disposed distal to the shroud and FIG. 18B shows a second aspect with the prosthetic heart valve disposed within the shroud.

FIG. 19 is a side view of an introducer sheath assembly.

FIG. 20 is an exploded perspective view of a loader assembly used for loading the balloon catheter and prosthetic valve into the introducer sheath assembly.

FIGS. 21A and 21B are side views illustrating the insertion of the delivery system into the loader assembly.

FIG. 22 is a side view illustrating the relationship between the delivery system, the introducer sheath assembly, and the loader assembly.

FIG. 23 is a side view of the delivery system during use, showing deployment of the prosthetic heart valve at the native valve site for replacing the function of a defective native valve.

FIGS. 24A and 24B are side views illustrating an example of a prosthetic valve that can be deployed using a delivery system of the present disclosure.

### DETAILED DESCRIPTION

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the disclosure is provided as an enabling teaching of the disclosure in its best, currently known aspect. To this end, those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and may even be desirable in certain circumstances and are a part of the present disclosure. Thus, the following description is again provided as illustrative of the principles of the present disclosure and not in limitation thereof.

### Definitions

As used in this application and in the claims, the singular forms “a,” “an,” and “the” include the plural forms unless the context clearly dictates otherwise. Thus, for example, reference to a “sleeve” includes aspects having two or more such frames unless the context clearly indicates otherwise.

It is also to be understood that the terminology used herein is for the purpose of describing particular aspects only and is not intended to be limiting. As used in the specification and in the claims, the term “comprising” can include the aspects “consisting of” and “consisting essentially of.”

Unless defined otherwise, all technical and scientific terms used herein have the same meaning as commonly understood by one of ordinary skill in the art to which this disclosure belongs. In this specification and in the claims, which follow, reference will be made to a number of terms that shall be defined herein.

For the terms “for example” and “such as,” and grammatical equivalences thereof, the phrase “and without limitation” is understood to follow unless explicitly stated otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 4%, within 3%, within 2%, with 1%, or within 0.5%) of the particular value modified by the term “about.”

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”). As used herein, the term “and/or” includes any and all combinations of one or more of the associated listed items.

It will be understood that, although the terms “first,” “second,” etc., may be used herein to describe various elements, components, regions, layers, sections, and/or steps. These elements, components, regions, layers, sections, and/or steps should not be limited by these terms. These terms are only used to distinguish one element, component, region, layer, section or steps from another element, component, region, layer, a section, or a step. Thus, a first element, component, region, layer, section or step discussed below could be termed a second element, component, region, layer, section, or step without departing from the teachings of exemplary aspects.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

As used herein, the term “substantially” means that the subsequently described event or circumstance completely occurs or that the subsequently described event or circumstance generally, typically, or approximately occurs.

Still further, the term “substantially” can in some aspects refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

As used herein, the term “substantially,” in, for example, the context “substantially no change” refers to a phenomenon or an event that exhibits less than about 1% change, e.g., less than about 0.5%, less than about 0.1%, less than about 0.05%, or less than about 0.01% change.

As used herein, the term “substantially,” in, for example, the context “substantially identical” or “substantially similar” refers to a method or a system, or a component that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

As used herein, the term “atraumatic” is commonly known in the art and refers to a device or a procedure that minimized tissue injury.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

Although the operations of exemplary aspects of the disclosed method may be described in a particular, sequential order for convenient presentation, it should be understood that disclosed aspects can encompass an order of operations other than the particular, sequential order disclosed. For example, operations described sequentially may, in some cases, be rearranged or performed concurrently. Further, descriptions and disclosures provided in association with one particular aspect are not limited to that aspect and may be applied to any aspect disclosed.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

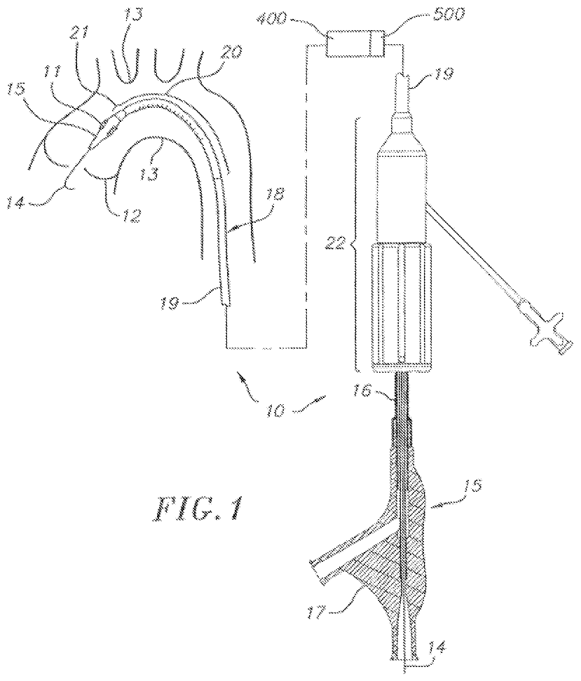

An exemplary delivery system is shown in FIG. 1. FIG. 1, for purposes of illustration, shows an aspect of a heart valve delivery system 10 for delivering a prosthetic valve 11 to a diseased aortic valve 12 of a human heart is shown. The delivery system is well-suited for delivering the prosthetic valve 11 through a patient's vasculature and over an aortic arch 13 to a location adjacent to the diseased valve 12.

The delivery system 10 generally includes a guidewire 14 and a balloon catheter 15 configured for advancement over the guidewire 14. The prosthetic valve 11 is provided along the distal end portion of the balloon catheter. The balloon catheter 15 includes a tubular section 16 and a handle/support 17 at a proximal end of the tubular section 16. The tubular section 16 of the balloon catheter 15 is received within a delivery sleeve assembly 18. The delivery sleeve assembly generally comprises an elongated polymeric sleeve 19 as disclosed in detail herein, a steerable section 20, and a shroud section 21. A proximal end of the delivery sleeve assembly 18 is mounted to a handle 22. The delivery system 10 passes through an introducer sheath assembly 400 and a loader assembly 500, both of which will be described in more detail below, to enter the body vessel and deliver the valve 11.

The following figures demonstrate described herein aspects in more detail. Additional exemplary aspects can also be found in U.S. Pat. Nos. 7,780,723, 8,382,826, 9,028,545, 9,907,651, and U.S. Pat. No. 10,478,294, contents of which are incorporated herein by reference in their whole entirety.

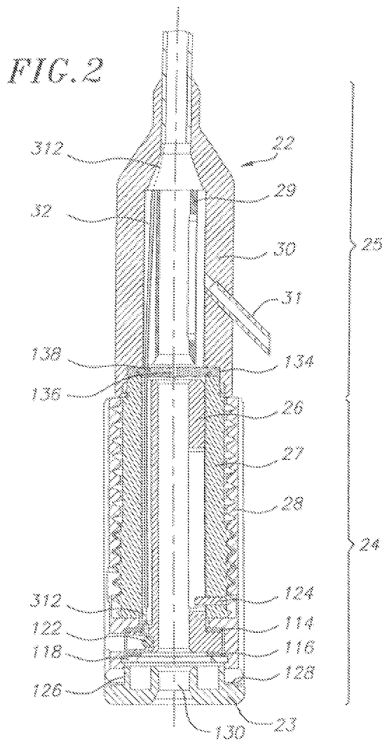

With reference to FIG. 2, the handle 22 at the proximal end of the delivery sleeve assembly 18 generally includes an end cap 23, an adjustable portion 24, and a hemostasis portion 25. The adjustable portion 24 includes a first core member 26, a partially threaded member 27 around the first core member 26, and a rotator handle 28 around the partially threaded member 27. The hemostasis portion 25 includes a second core member 29 and a hub 30 around the second core member 29. A hemostasis tube 31 extends outwards from the hub 30. A guide tube 32 is placed within the handle 22, as described in greater detail below.

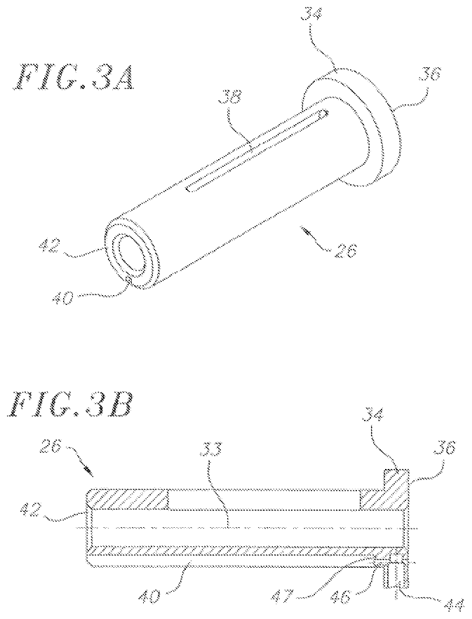

With reference to FIGS. 3A and 3B, the first core member 26, is generally tube-shaped, having a passageway 33 extending longitudinally therethrough. An annular flange 34 forms a proximal end 36 of the first core member 26. A first slot opening 38 allows communication from the outer surface of the first core member 26 into the passageway 33 and along a length of the first core member 26. A second slot 40 travels along the length of the outer surface of the first core member 26 from a distal end 42 towards the flange 34. The flange 34 includes a first fastener opening 44 extending radially from the outer surface of the first core member 26. A longitudinally extending access opening 46 at a proximal end of the slot 40 extends from a proximal end wall 47 of the slot 40 into the first fastener opening 44.

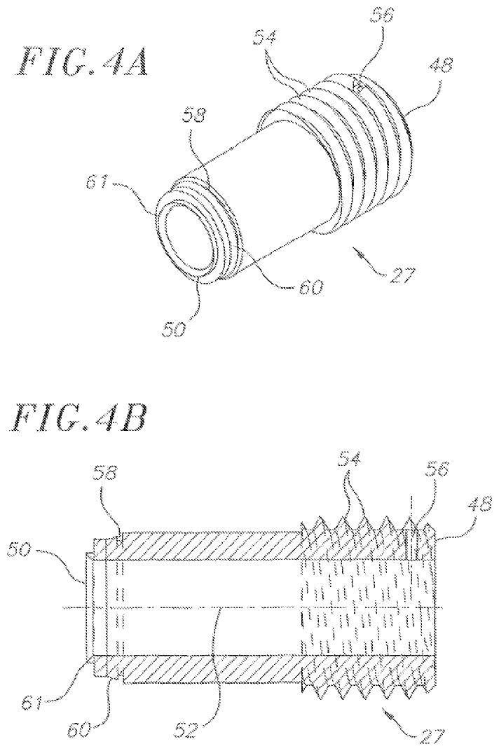

With reference to FIGS. 4A and 4B, the partially threaded member 27 has a proximal end 48 and a distal end 50. The partially threaded member 27 is generally tube-shaped, having a passageway 52 extending longitudinally therethrough. Toward the proximal end 48, the outer surface of the partially threaded member 27 has an exterior thread 54. The thread 54 includes a radially extending dowel opening 56 extending into the passageway 52 of the partially threaded member 27. Toward the distal end 50, the outer surface of the partially threaded member 27 forms an annularly shaped groove 58. The outer surface of the partially threaded member 27 also forms a tapered surface 60, located distally adjacent to the annularly shaped groove 58, toward the distal end 50. A pointed annular tip 61 forms the distal end 50 of the partially threaded member 27.

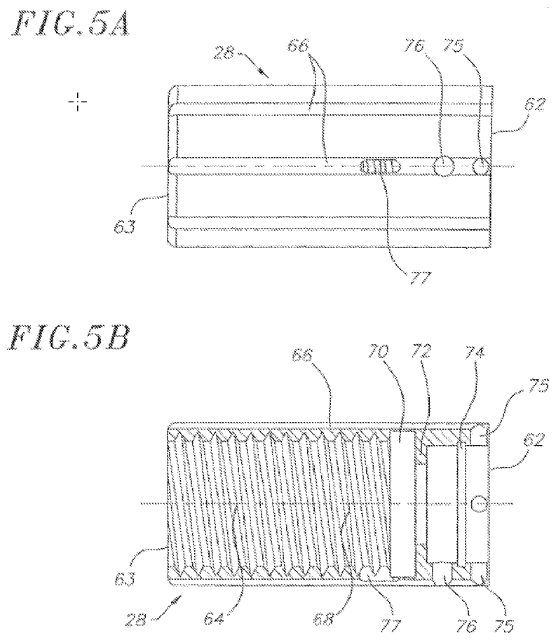

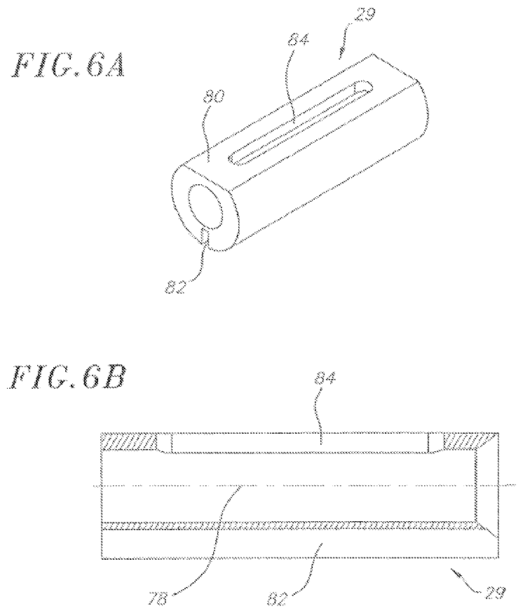

With reference to FIGS. 5A and 5B, the rotator handle 28 can comprise an elongated cylinder having a proximal end 62 and a distal end 63 and includes a passageway 64 extending longitudinally therethrough. On its outer surface, the rotator handle 28 includes grooved portions 66 extending along its length. On its inner surface, the rotator handle 28 includes a threaded portion 68 that extends inwardly from the distal end 63, a first annularly shaped recess 70 proximally adjacent the threaded portion 68, an annular flange 72 adjacent the first annularly shaped recess 70 extending inwardly from the inner surface, and a second annularly shaped recess 74 adjacent the proximal end 62 of the rotator handle 28. Fastener openings 75 pass from the outer surface to the inner surface of the rotator handle 28 in the area of the passageway 64 located proximally adjacent the second annularly shaped recess 74 distally adjacent the proximal end 62 of the rotator handle 28. An access opening 76 passes from the outer surface to the inner surface of the rotator handle 28 in the area of the passageway 64 distally adjacent the second annularly shaped recess 74 and proximally adjacent the annularly shaped flange 72. A second access opening 77 also extends from the outer surface to the inner surface of the rotator handle 28 at a proximal end of the threaded portion 68.

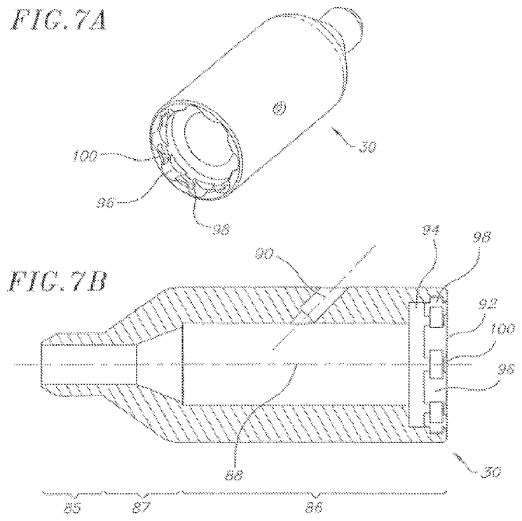

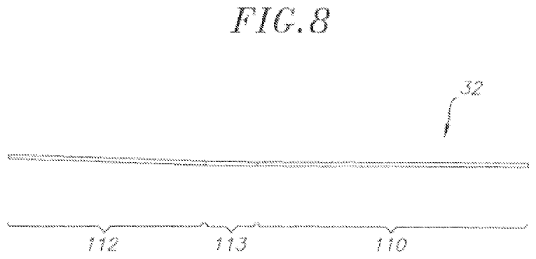

With reference to FIGS. 6A and 6B, the second core member 29, is generally tube-shaped and includes a passageway 78 extending therethrough. A flat portion 80 of the second core member 29 further defines its outer surface. The outer surface of the second core member 29 includes a slot 82, which travels longitudinally along its length. The second core member 29 also includes a longitudinally extending slot 84 passing through the flat portion 80 of the outer surface into the passageway 78 of the second core member 29.

With reference to FIGS. 7A and 7B, the hub 30 is formed by first and second cylindrical sections 85, 86 connected by a tapered section 87. A passageway 88 extends through the hub 30. The passageway 88 increases in size in the tapered section 87 while transitioning from the first cylindrical section 85 to the second cylindrical section 86. A hemostasis valve opening 90 extends diagonally from an outer surface of the second cylindrical section 86 to an inner surface thereof. At a proximal end 92 of the hub 30, the inner surface includes an annularly shaped principal recess 94 that forms a shoulder at a proximal end of the passageway 88. Additional semi-cylindrical recesses 96 are located around the circumference of the annularly shaped principal recess 94. A second annularly shaped recess 98 extends around the inner surface of the hub 30 in the area in which the semi-cylindrical recesses 96 are located, leaving individual flanges 100 extending radially inwardly along the inner surface at the proximal end 92 of the hub 30.

The guide tube 32, shown in FIG. 8, is tube-shaped and has a passageway extending longitudinally therethrough. A proximal section 110 and a distal section 112 are both straight and form an angled relation to each other. A transition section 113 is curved and connects the proximal and distal sections 110, 112.

The component parts of the handle 22 can be assembled, as shown in FIG. 2. A first thrust washer 114 is placed on the outer surface of the first core member 26 distally adjacent the flange 34 (see FIG. 3A) of the first core member 26, and the first core member 26 is inserted into the rotator handle 28 through the proximal end 62 (see FIG. 5A) of the rotator handle 28. A second thrust washer 116 is placed proximal to the proximal end 36 of the first core member 26. The first thrust washer 114 is sandwiched between the annular flange 72 of the rotator handle 28 and the flange 34 of the first core member 26. The flange 34 sits in the area between the annularly shaped flange 72 and the second annularly shaped recess 74 of the rotator handle 28. A snap ring 118 is placed in the second annularly shaped recess 74 (see FIG. 5B) and contacts the second thrust washer 116, thus retaining the position of the first core member 26.

A first core member fastener (not shown) engages the first fastener opening 44 (see FIG. 3B) of the first core member 26. A ball bearing 122 is placed in the first fastener opening 44. The access opening 76 (see FIG. 5B) of the rotator handle 28 allows for access to the first core member fastener.

The partially threaded member 27 is screwed into the rotator handle 28 from the distal end 63 of the rotator handle 28. The exterior thread 54 of the partially threaded member 27 engages the threaded portion 68 of the inner surface of the rotator handle 28. The first core member, 26, sits inside the passageway 52 of the partially threaded member 27. When the partially threaded member 27 is fully engaged within the rotator handle 28, as shown in FIG. 2, the proximal end 48 of the partially threaded member 27 abuts the annularly shaped flange 72 of the rotator handle 28.

A dowel 124 engages the dowel opening 56 of the partially threaded member 27 (see FIG. 4B) and extends from the outer surface of the partially threaded member 27 into the first slot opening 38 of the first core member 26. When the partially threaded member 27 is fully engaged in the rotator handle 28, the dowel 124 is located in the area of the passageway 64 of the rotator handle 28, corresponding to the first annularly shaped recess 70 (see FIG. 5B). The dowel 124 is placed into the dowel opening 56 of the partially threaded member 27 through the second access opening 77 of the rotator handle 28 as the partially threaded member 27 is screwed into the rotator handle 28 and the dowel opening 56, second access opening 77, and the first slot opening 38 of the first core member 26 are aligned.

The end cap 23 is secured to the proximal end 62 of the rotator handle 28. The end cap 23 includes a cylindrically shaped first contact surface 126, which contacts the inner surface of the rotator handle 28 and a second contact surface 128, which contacts the proximal end 62 of the rotator handle 28. A passageway 130 extends through the end cap 23 and is placed in communication with the passageway 64 of the rotator handle 28. The first contact surface 126 of the end cap 23 is aligned with the fastener openings 75 of the rotator handle 28. Set screws (not shown) engage the fastener openings 75 to secure the end cap 23 to the rotator handle 28.

The second core member, 29, is placed in passageway 88 of the hub 30. The slot opening 84 (see FIG. 6B) of the second core member 29 is aligned with the hemostasis valve opening 90 (see FIG. 7B) of the hub 30. A slab 134 is placed in the annularly shaped principal recess 94 of the hub 30 proximally adjacent to the second core member 29. The slab 134 can comprise polyisoprene and includes a central opening 136 placed in communication with the passageway 88 of the second core member 29 as well as a guide tube opening 138, which is placed in communication with the slot 82 of the second core member 29. The slab 134 can be adhered to the inner surface of the hub 30.

The proximal section 110 (see FIG. 8) of the guide tube 32 is inserted into the slot 40 of the first core member 26. The guide tube 32 passes through the slab 134. The distal section 112 of the guide tube 32 is inserted into the slot 82 of the second core member 29.

The pointed annular tip 61 (see FIG. 4B) of the partially threaded member 27 is pressed into the slab 134, and the individual flanges 100 (see FIG. 7A) at the proximal end 92 of the hub 30 engage in the annularly shaped groove 58 of the partially threaded member 27 to connect the hub 30 to the partially threaded member 27. The flanges 100 ride along the tapered surface 60 of the partially threaded member 27 before engaging the annularly shaped groove 58 of the partially threaded member 27. Upon assembly between the partially threaded member 27 and the hub 30, and when the partially threaded member 27 is fully engaged in the rotator handle 28, the proximal end 92 of the hub 30 abuts the rotator handle 28. Further, as shown in FIG. 2, the center section 113 of the guide tube 32 passes through the slab 134.

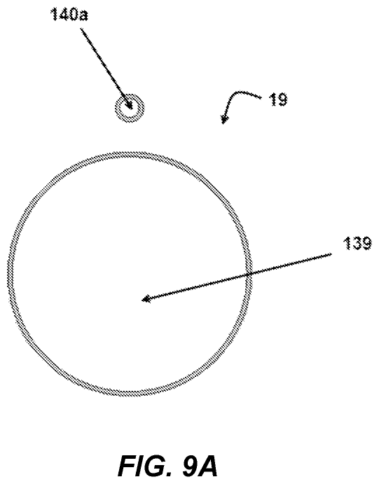

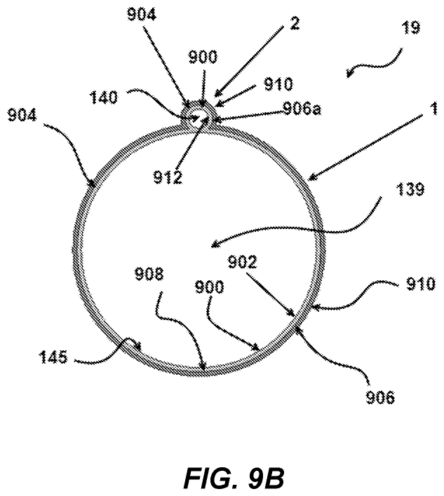

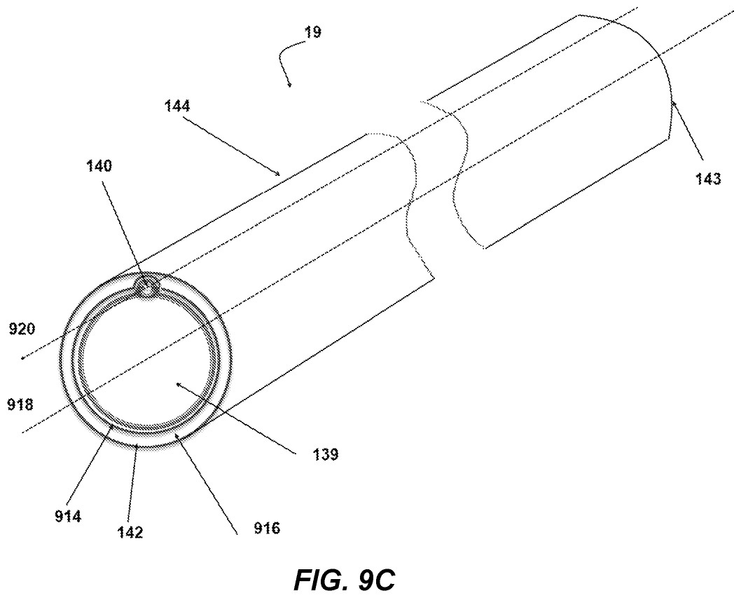

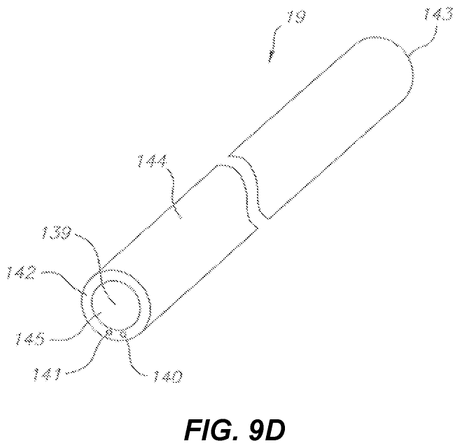

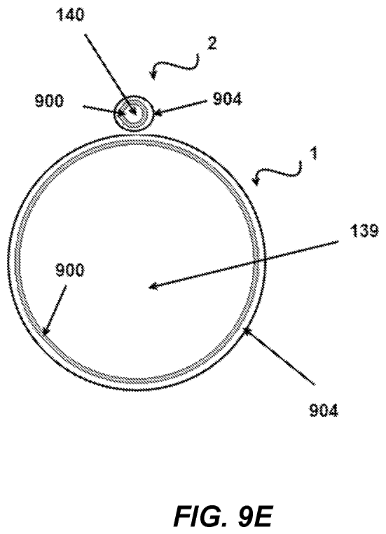

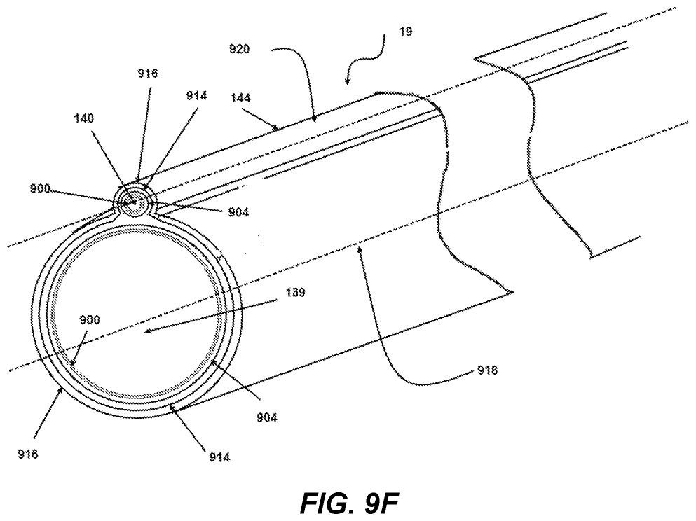

The aspects disclosed and claimed herein are illustrated in FIG. 9A-9F. Referring to FIG. 9A, this figure illustrates an aspect as used in the prior art. In such aspects, the delivery system comprises two elongated sleeves, one having a central lumen 139 and one having an additional lumen 140. In this aspect, the central lumen 139 is configured to accommodate a balloon catheter, while the lumen 140 is configured to accommodate a pull wire. In such known aspects, the materials used to form these two lumens often comprise PTFE-based materials. In certain aspects, these PTFE-based materials can also be etched to improve the lubricating properties of the inner surfaces. While in other aspects, these PTFE-based materials can also be etched to improve adhesion/thermal bonding of the PTFE to other plastics. It is also understood, however, that PTFE in certain aspects can exhibit poor adhesion to additional layers of the sleeve and cause delamination and, therefore, failure of the system. Thus, solutions to avoid such failures are explored in the disclosed herein sleeves. An additional challenge of currently known systems is to ensure a substantially parallel alignment between both lumens along the whole length. Any deviations can cause undesirable effects and make the delivery of catheters significantly more difficult and more dangerous to the patient.

FIGS. 9B and 9C disclose an elongated polymeric sleeve 19 having a proximal end 142 and a distal end 143 (FIG. 9C) that comprises an inner polymer layer 900 and an outer polymer layer 904. As can be seen in FIG. 9B the inner polymer layer of the elongated polymeric sleeve forms a first tubular sleeve 1 having an inner surface 902 defining a first lumen 139 having a first diameter, an outer surface 906, and a first longitudinal axis 918 (as shown in FIG. 9C). The inner polymer layer 900 of the elongated polymeric sleeve also forms a second tubular sleeve 2 that is permanently adjacent to the first tubular sleeve, wherein the second tubular sleeve has an inner surface 912 defining a second lumen 140 having a second diameter, and an outer surface 906a that is an extension of the outer surface 906 of the first tubular sleeve and wherein the second tubular sleeve has a second longitudinal axis 920 (as shown in FIG. 9C). It can be seen that the second diameter is substantially smaller than the first diameter. The first lumen 139 is configured to deliver a balloon catheter, as discussed in detail below, while the second lumen 140 is configured to pass a pull wire.

In still further aspects and as shown in FIG. 9C, the second longitudinal axis 920, can be offset of the first longitudinal axis 918 while keeping a substantially parallel alignment to the first longitudinal axis 918 along a length of the elongated polymeric sleeve. It can also be seen that the outer surface 906 of the first tubular sleeve and the second tubular sleeve 906a define an outer surface of the inner polymer layer. Also, the inner surface of the first tubular sleeve 902 defines an inner surface 145 of the elongated polymeric sleeve.

Further, as shown in FIG. 9B, the outer polymer layer 904 extends circumferentially around the inner polymer layer, wherein the outer polymer layer has an inner surface 908 and an outer surface 910. As disclosed in these exemplary aspects, the inner surface 908 of the outer polymer layer is adjacent and bonded to the outer surface 906, 906a of the inner polymer layer.

An alternative configuration of the sleeve is shown in FIGS. 9E and 9F. In such aspects, the first and the second sleeves can be formed separately. This can be advantageous in the aspects where the delivery apparatus does not require the presence of a pull wire. However, in aspects where the delivery apparatus requires the presence of the pull wire, two tubular sleeves 1 and 2 are separately formed and arranged as desired for a specific application. Sleeve 1 is formed to have a first lumen 139, while sleeve 2 is formed to have a second lumen 140. Since sleeve 1 is configured to accommodate a balloon catheter, its lumens' (139) diameter is substantially larger than the diameter of lumen 140 of the sleeve 2 that is configured to accommodate a pull wire. Each sleeve has an inner polymer layer 900 and an outer polymer layer 904 (FIG. 9E). The two sleeves are formed separately and substantially aligned with each other.

In further aspects, the braided layer 914 is disposed at the outer polymer layer 904. When both tubular sleeves are present, the braided layer is disposed such that it encompasses both the first and the second tubular sleeves. In yet further aspects, an additional outer jacket 916 is disposed on the braided layer 914 such that it again encompasses both the first tubular sleeve and the second tubular sleeve. All construction is then fused to allow inseparable coupling between the two sleeves. It is understood that such a configuration results in the second tubular sleeve being fixated along a length of the first tubular sleeve allowing substantially parallel alignment between the sleeves.

In still further aspects and as shown in FIG. 9F, the second longitudinal axis 920, can be offset of the first longitudinal axis 918 while keeping a substantially parallel alignment to the first longitudinal axis 918 along a length of the elongated polymeric sleeve. In an alternative aspect, as shown in FIG. 9D the elongated polymeric sleeve 19 can be formed with a first lumen 139 and a second lumen 140, and a third lumen 141. It is understood that when two lumens are present in addition to the first lumen 139, the second and the third lumens can be formed by any methods known in the art. In certain aspects, the first lumen 139, the second lumen 140, and the third lumen 141 can be formed by co-extrusion, similarly to the elongated polymer sleeve 19 shown in FIG. 9B. Yet, in other aspects, the second lumen 140 and the third lumen 141 can be formed similar to the aspects shown in FIGS. 9E-9F.

In further aspects, the inner polymer layer can form the first tubular sleeve 139 having a first diameter, the second tubular sleeve 140 having a second diameter, and a third tubular sleeve 141 having a third diameter. In such aspects, the first diameter is substantially larger than the second and/or the third diameters. In still further aspects, the second and third diameters can be the same or different. It is further understood that the second and the third tubular sleeves can be positioned adjacent to each other or spaced from each other as desired. In yet still further aspects, the third tubular lumen can have a third longitudinal axis that is offset of the first longitudinal axis and is substantially parallel to the first longitudinal axis. In still further and unlimiting aspects, the second and the third longitudinal axis can also be parallel to each other. It is also understood that these aspects are only exemplary, and if desired or required, the elongated polymeric sleeve can comprise three or more tubular sleeves. In such aspects, all of the tubular sleeves can be co-extruded and comprise any of the materials disclosed herein.

Referring back to FIG. 9D, the elongated polymeric sleeve also includes a proximal end 142 and a distal end 143, an outer surface 144, and an inner surface 145. As discussed above, this exemplary sleeve can be formed from any suitable materials. In some exemplary aspects, this elongated polymeric sleeve is formed from any materials disclosed for the sleeve shown in FIGS. 9B and 9C.

As disclosed in this exemplary aspect, the first and the second tubular sleeves are attached permanently. Such an attachment is achieved by the methods disclosed herein that comprise co-extruding the inner polymer layer and the outer polymer layer to form the disclosed sleeve. In still further aspects, any materials known in the art can be used to form the inner and the outer polymer layers of the elongated polymeric sleeve.

In still further aspects, the inner polymer layer can have a thickness from about 1 to about 5 mils, including exemplary values of about 1.2 mils, about 1.5 mils, about 1.7 mils, about 2 mils, about 2.2 mils, about 2.5 mils, about 2.7 mils, about 3 mils, about 3.2 mils, about 3.5 mils, about 3.7 mils, about 4 mils, about 4.2 mils, about 4.5 mils, and about 4.7 mils. It is understood that the inner layer can have any thickness value between any two foregoing values.

In yet further aspects, the outer polymer layer can have a thickness from about 1 to about 5 mils, including exemplary values of about 1.2 mils, about 1.5 mils, about 1.7 mils, about 2 mils, about 2.2 mils, about 2.5 mils, about 2.7 mils, about 3 mils, about 3.2 mils, about 3.5 mils, about 3.7 mils, about 4 mils, about 4.2 mils, about 4.5 mils, and about 4.7 mils. It is understood that the outer polymer layer can have any thickness value between any two foregoing values. It is also understood that in some aspects, the inner polymer layer has a thickness that is substantially similar to a thickness of the outer polymer layer. While in other aspects, the inner polymer layer has a thickness that is different from a thickness of the outer polymer layer. In some aspects, the inner layer can have a thickness that is larger than a thickness of the outer polymer layer, while in other aspects, the inner layer can have a thickness that is smaller than a thickness of the outer polymer layer.

In certain aspects, the inner polymer layer comprises a first polymer, while in other aspects, the outer polymer layer comprises a second polymer. In certain aspects, the first polymer can comprise a fluoropolymer. Any known in the art fluoropolymers that can be extruded with other desired polymers to provide a strong bond can be utilized. In still further exemplary and unlimiting aspects, a fluoropolymer such as Daikin EFEP RP-5000 or Daikin RP-4020 can be utilized. In yet still further aspects, the fluoropolymer can comprise a copolymer of ethylene-perfluoroethylenepropene. It is understood, however, that the disclosed above fluoropolymers are only exemplary and non-limiting, and any fluoropolymers that can be coextruded with different polymers without delamination and useful for the disclosed herein application can be utilized. In yet other aspects, the second polymer can comprise a polyamide, a polyether block amide, or a combination thereof. In still further aspects, the polyamide can comprise a nylon 12. While in other aspects, the polyether block amide can comprise PEBAX® provided by Arkema having various durometers. While in still further aspects, the polyether block amide can comprise PEBA-B provided by Evonik. In still further aspects, the outer polymer layer can comprise nylon-12 and/or PEBA-B.

In certain aspects, the outer polymer layer near the distal end of the elongated polymeric sleeve comprises a durometer different from the outer polymer layer near the proximal end of the elongated polymeric sleeve. It is understood that in aspects where two tubular sleeves are not formed by coextrusion, for example, in the aspects shown in FIGS. 9E and 9F, the elongated polymeric sleeve references to a sleeve that contains either the first tubular sleeve, or the second tubular sleeve, or both the first and the second tubular sleeves together. Toward the distal end, the elongated polymeric sleeve 19, the outer polymer layer can include a soft durometer section capable of flexing. In the aspects where the outer polymer layer comprises PEBAX®, the soft durometer section of the elongated polymeric sleeve 19 (or the first and the second tubular sleeves if the formed separately) can be made of 55D PEBAX® and is capable of flexing; while a remaining portion of the elongated polymeric sleeve 19 as shown in the exemplary aspect can be made of 72D PEBAX®, which is stiffer than 55D PEBAX®. Without wishing to be bound by any theory, the stiffness of 72D PEBAX® can prevent the elongated polymeric sleeve from excessive bending, thus giving the operator the ability to push the delivery system 10 through the potentially constricting body vessel and allowing the delivery system 10 to more effectively track to the native valve site, as described below. The elongated polymeric sleeve 19 can also be formed of wire braid anywhere along the length thereof. Wire braid can also contribute to the stiffness and pushability of the delivery system 10. A similar approach can be taken with the elongated polymeric sleeve 19, where the outer polymer layer comprises nylon-12 or PEBA-B. In such exemplary aspects, a distal portion of the elongated polymeric sleeve 19 can comprise a polymer exhibiting a softer durometer, while other portions of the elongated polymeric sleeve can comprise a polymer exhibiting a firmer durometer.

In still further aspects and as shown in FIG. 9C, the elongated polymeric sleeve, can further comprise an outer jacket 916. Again, it is understood that the elongate polymeric sleeve can refer to co-extruded dual-lumen tubular sleeves, or to the first tubular sleeve and/or the second tubular sleeve, if they are formed separately, or to the elongated sleeve when the first and the second tubular sleeves are aligned with each other. In certain aspects, the outer jacket can be disposed over the outer surface 910 of the outer polymer layer of the elongated polymeric sleeve. While in still further aspects, the elongated polymeric sleeve can further comprise a braided layer 914 disposed between the outer jacket and the outer surface of the outer polymer layer of the elongated polymeric sleeve. In still further aspects, the inner and outer polymer layers of the elongated polymeric sleeve and/or braided layer and/or the outer jacket are fused together.

It is understood that the outer jacket can comprise any materials that can provide for the desired result. In certain aspects, the outer jacket can comprise a polyether block amide, such as PEBAX®. In yet further aspects, the braided layer can comprise any known in the art materials. In certain aspects, the braided layer can comprise a metal mesh, while in other aspects, it can comprise a laser cut hypo tube.

Further, the delivery systems that are disclosed herein can comprise a selectively steerable section having a proximal end and a distal end, wherein the proximal end of the steerable section is permanently coupled to the distal end of the elongated polymeric sleeve; wherein the steerable section comprises a central lumen having an inner diameter; wherein the central lumen is coaxial with the first lumen of the first tubular sleeve; and wherein the selectively steerable section is substantially more flexible than the elongated polymeric sleeve, and wherein the selectively steerable section is configured to provide a sufficient curvature to navigate around an aortic arch.

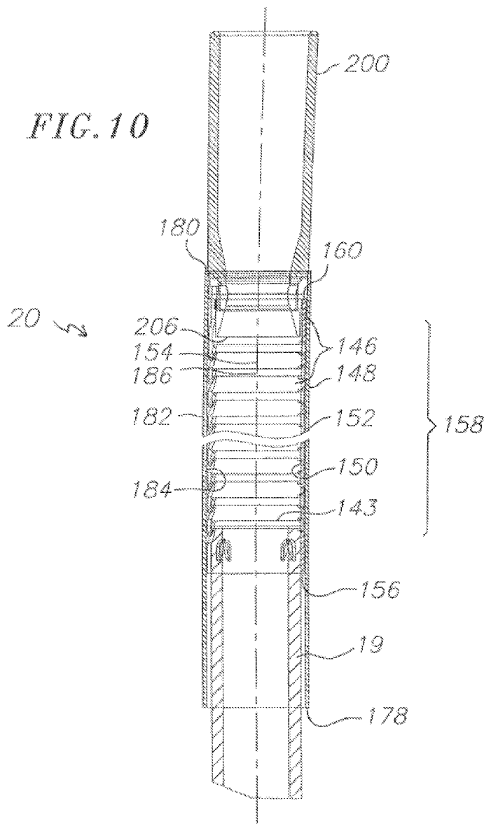

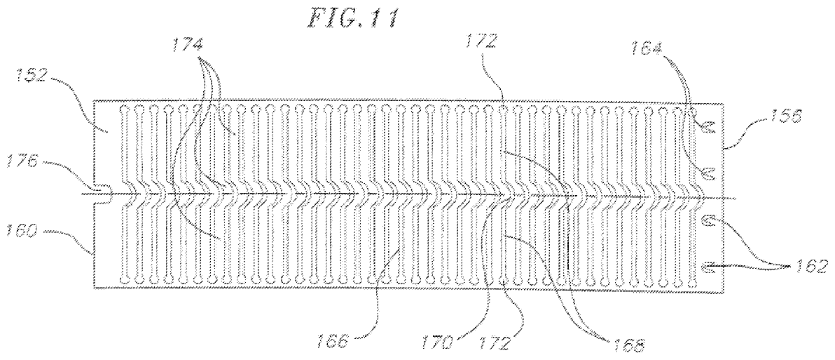

FIG. 10 shows the steerable section 20 of the delivery sleeve assembly in cross-section. The steerable section generally includes a flex tube 146 and a cover 148. The flex tube 146 can be tube-shaped, for example, having an inner surface 150, an outer surface 152, and a passageway 154 extending therethrough. The flex tube 146 is further defined by a proximal end 156, a center section 158, and a distal end 160. With reference to FIG. 11, a plurality of V-shaped notches 162 are provided, such as by laser cutting, in the flex tube 146 adjacent the proximal end 156. The notches 162 are shaped to provide pointed barbs 164. Along the center section 158 of the flex tube 146, circumferentially extending elongate openings 166 are provided. Each elongate opening 166 can include two elongate portions 168 connected by a curved portion 170. Circular portions 172 are provided at the ends of the elongate openings. Tube portions 174 remain substantially intact and will be described in more detail below. A notch 176 is formed at the distal end 160 of the flex tube 146. In one aspect, the flex tube 146 is made of a stainless-steel hypo-tube.

With reference again to FIG. 10, the cover 148 can be tube-shaped, for example, having proximal and distal ends 178, 180, and including an outer surface 182 and an inner surface 184, with a passageway 186 extending longitudinally therethrough. In one aspect, the cover 148 is formed of soft durometer material such as 55D PEBAX®. The soft durometer 55D PEBAX® of the cover 148 allows it to stretch and flex, as described below.

The steerable section 20 is assembled by placing the flex tube 146 inside the cover 148. The cover 148 may be stretched prior to assembly to give the steerable section 20 desirable features, as outlined below. The outer surface of the flex tube 146 contacts the inner surface of the cover 148. The proximal end 178 of the cover 148 extends proximally from the proximal end 156 of the flex tube 146, and the distal end 180 of the cover 148 extends distally from the distal end 160 of the flex tube 146.

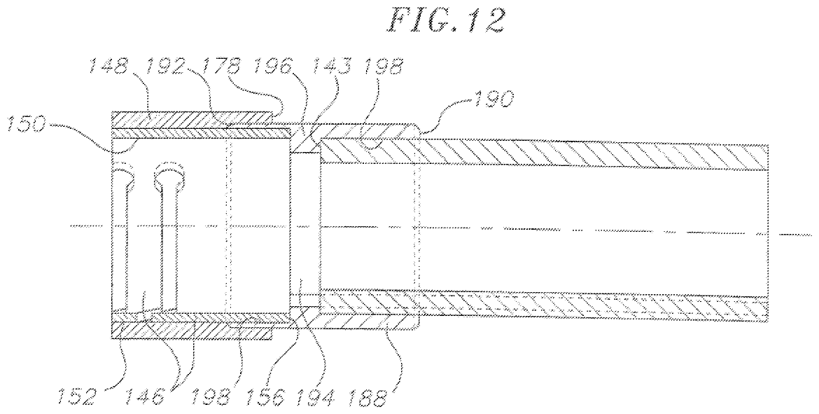

With reference to FIG. 12, an alternative aspect of the steerable section 20 includes a connector 188 having a proximal end 190 and a distal end 192.

The connector 188 is tube-shaped, having a passageway 194 longitudinally extending therethrough. An annularly shaped flange 196 protrudes from an inner surface 198 of the connector 188.

To assemble the alternative aspect of the steerable section 20, including the connector 188, the proximal end 156 of the flex tube 146 is inserted into the passageway 194 of the connector 188 until it abuts the annularly shaped flange 196. The outer surface 152 of the flex tube 146 contacts the inner surface 198 of the connector 188 and can be adhered thereto using adhesion. The cover 148 is placed over the flex tube 146 and the connector 188. The proximal end 190 of the connector 188 extends proximally from the proximal end 178 of the cover 148, and the distal end 180 of the cover 148 extends distally from the distal end 160 of the flex tube 146 (see FIG. 10).

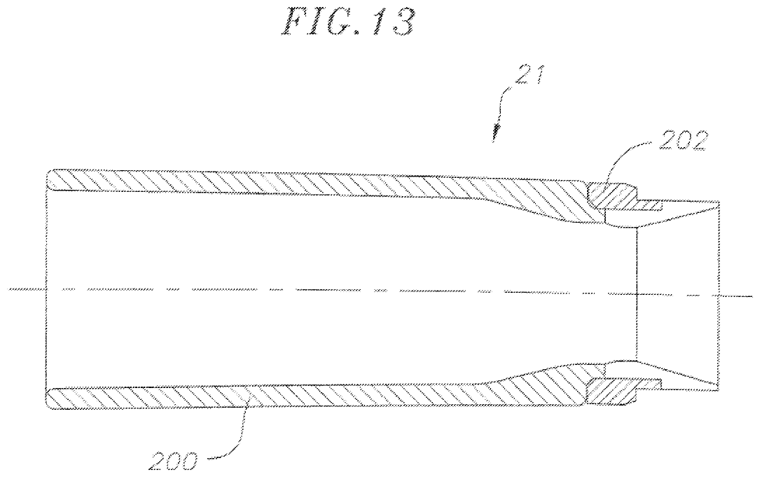

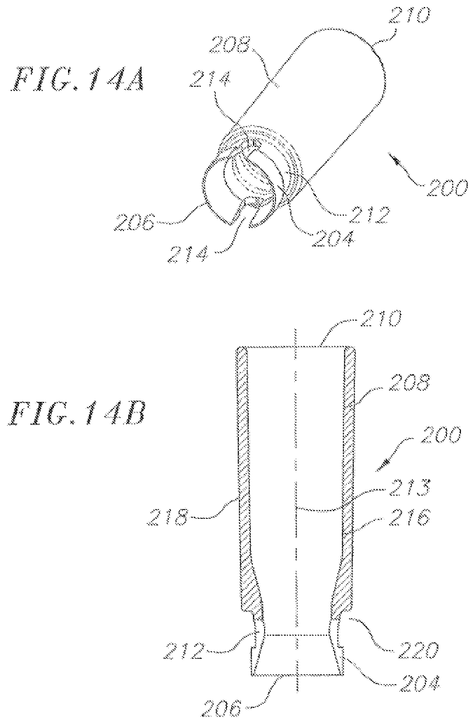

With reference to FIG. 13, the shroud section 21 is shown in cross-section. The shroud section 21 generally includes a shroud 200 and a ring 202. With reference to FIGS. 14A and 14B, the shroud 200 can be cylindrical-shaped and comprises three continuous cylindrical sections: a rim 204 near a proximal end 206, a main body 208 near a distal end 210, and a neck 212 located therebetween. A passageway 213 extends through the shroud 200, which includes an inner surface 216 and an outer surface 218. Slots 214 run from the proximal end 210 of the shroud 200 into the neck 212. The neck 212 has a smaller circumference than the rim 204 and the main body 208, resulting in a groove 220 along the outer surface 218 of the shroud 200.

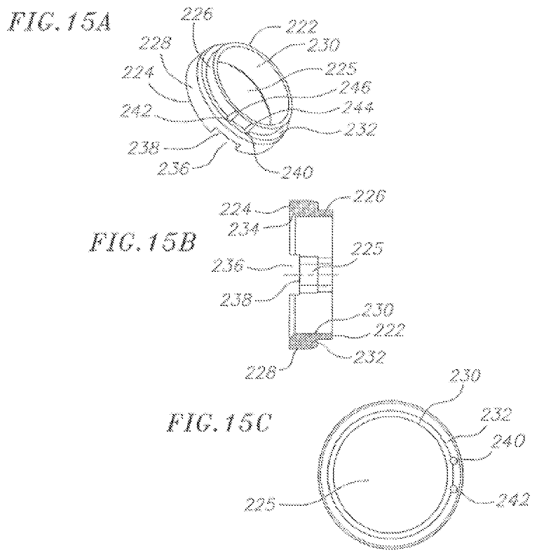

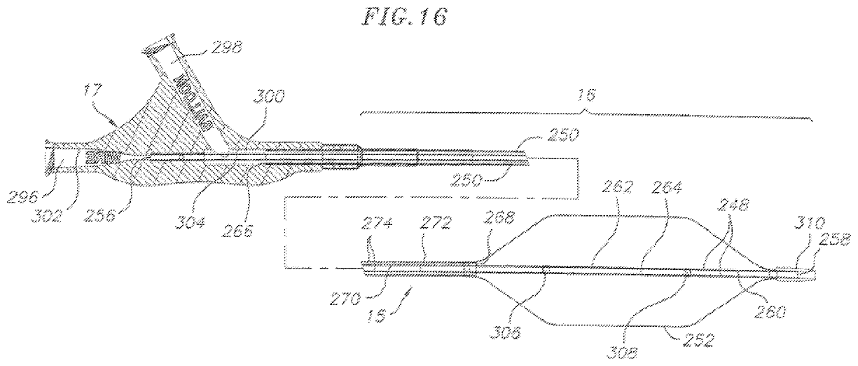

With reference now to FIGS. 15A through 15C, the ring 202 has a proximal end 222, a distal end 224, and a passageway 225 extending longitudinally therethrough. The ring 202 includes a proximal outer surface 226, a distal outer surface 228, and an inner surface 230. An outer face 232 runs perpendicular to the proximal and distal outer surfaces 226, 228 of the ring 202 and connects the proximal and distal outer surfaces 226, 228, which generally run parallel to one another. The inner surface 230 includes an angled surface 234 toward the distal end 224, causing the passageway 225 of the ring to increase in diameter near the distal end 224 of the ring 202.

A slot 236 extends into the distal end of the ring 202 and through the distal outer surface 228 to the inner surface 230 and parallel to a central axis of the ring 202, creating a slot face 238 opposed to the outer face 232. A first lumen 240 and a second lumen 242 extend from the slot face 238 to the outer face 232 of the ring 202. The proximal outer surface 226 also includes a first semi-cylindrical recess 244 and a second semi-cylindrical recess 246, which run parallel to the central axis of the ring 202 and pass from the proximal end 222 to the outer face 232 of the ring 202. The first cylindrical recess 244 is aligned with the first lumen 240, and the second cylindrical recess 246 is aligned with the second lumen 242.

The shroud section 21 is formed by inserting the proximal end of the shroud 200 into the ring 202, according to FIG. 13. The rim 204 flexes to permit this. The ring 202 fits snugly in the groove 220 (see FIG. 14B), such that the inner surface 230 and the proximal and distal ends 222, 224 of the ring 202 (see FIG. 15A) contact the outer surface 218 of the shroud 200. The ring 202 is situated so that either of the slots 214 of the shroud 200 (see FIG. 14A) is aligned with the slot 236 of the ring 202.

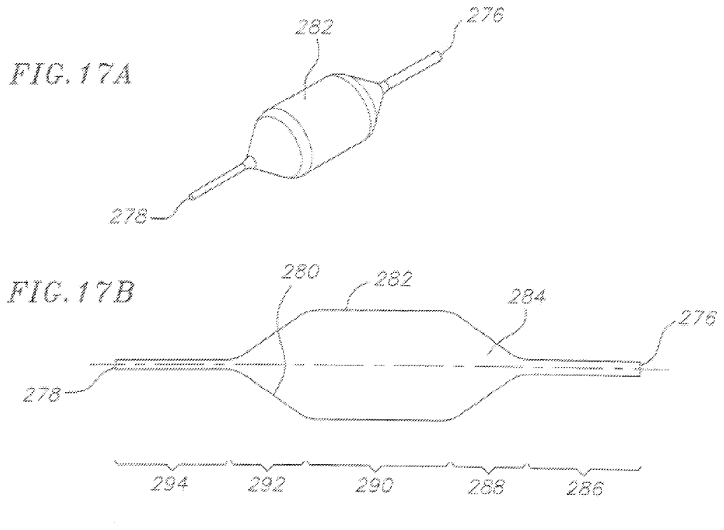

With reference to FIG. 16, the balloon catheter 15 includes a tube section 16 and a support 17. The tube section 16 includes a guidewire shaft 248, a balloon shaft 250, both of which are connected to the support 17, and a balloon 252. The guidewire shaft 248, having a proximal end 256 and a distal end 258, includes an inner surface 260, an outer surface 262, and a passageway 264 longitudinally extending therethrough. The guidewire shaft 248 can be formed of nylon, braided stainless steel wires, or PEBAX® at differing portions along its length, according to the need for rigidity and flexibility. Teflon® can be used to form the inner surface 260 of the guidewire shaft 248. The balloon shaft 250, having a proximal end 266 and a distal end 268, includes an inner surface 270, an outer surface 272, and a passageway 274 longitudinally extending therethrough. The balloon shaft 250 can be formed of any combination of nylon, PEBAX®, or braided stainless steel wires at differing portions along its length, according to the need for rigidity and flexibility.

With reference now to FIGS. 17A and 17B, the balloon 252 has a proximal end 276 and a distal end 278 includes an inner surface 280, an outer surface 282, and a passageway 284 extending longitudinally therethrough. When viewed from the proximal end 276 to the distal end 278, the balloon 252 includes five portions: a first slender portion 286, a first cone portion 288, a main cylindrical portion 290, a second cone portion 292, and a second slender portion 294. The balloon 252 can be formed of nylon and is rated at a burst pressure of 6-8 atm, including exemplary values of about 6.5 atm, about 7 atm, and about 7.5 atm. In some aspects, the expanded diameter of the balloon ranges from about 20 to 28 mm, including exemplary values of about 21 mm, about 22 mm, about 23 mm, about 24 mm, about 25 mm, about 26 mm, and about 27 mm.

With reference again to FIG. 16, the support 17 includes a wire inlet opening 296, a fluid inlet opening 298, and a main shaft opening 300. The wire inlet opening 296 includes an interior surface 302, and the main shaft opening 300 likewise includes an interior surface 304. The openings 296, 298, 300 are arranged so as to be in communication with one another.

The balloon catheter 15 is assembled, as shown in FIG. 16. The guidewire shaft 248 is inserted into the main shaft opening 300. The proximal end of the guidewire shaft 248 is placed in the wire inlet opening 296, and the outer surface 262 of the guidewire shaft 248 is secured to the interior surface 302 of the wire inlet opening 296, for example, by adhesion. The guidewire shaft 248 is of a smaller diameter than the main shaft opening 300 and, as such, does not contact the interior surface 304 of the main shaft opening 300.

The balloon shaft 250 is placed over the guidewire shaft 248. The proximal end 266 of the balloon shaft 250 is placed in the main shaft opening 300 of the support 17, and the outer surface 272 of the balloon shaft 250 is secured to the interior surface 304 of the main shaft opening 300. As shown in FIG. 16, the guidewire shaft 248 is of a smaller diameter than the balloon shaft 250, and the outer surface 262 of the guidewire shaft 248 does not contact the inner surface 270 of the balloon shaft 250 to permit airflow.

The proximal end 256 of the guidewire shaft 248 extends proximally from the proximal end 266 of the balloon shaft 250, and the distal end 258 of the guidewire shaft extends distally from the distal end 268 of the balloon shaft 250.

The proximal end 276 of the balloon 252 is placed over the distal end 268 of the balloon shaft 250. The inner surface 280 of the balloon 252 in the area of the first slender portion 286 is secured to the outer surface 272 of the balloon shaft 250. The distal end 278 of the balloon 252 is placed over the distal end 258 of the guidewire shaft 248. The inner surface 280 of the balloon 252 in the area of the second slender portion 294 is secured to the outer surface 262 of the guidewire shaft 248. The balloon 252 can be secured to the balloon shaft 250 and the guidewire shaft 248 by a process involving the curing of adhesive with an ultraviolet light or laser welding.

First and second marker bands 306, 308 are placed along the guidewire shaft 248 within the passageway 284 of the balloon 252. The marker bands 306, 308 can be secured to the outer surface 262 of the guidewire shaft 248 by an adhesive or swaging. The position of the first marker band 306 roughly corresponds to the transition between the first cone portion 288 and the main cylindrical portion 290 of the balloon 252 (see FIG. 17B). The position of the second marker band 308 roughly corresponds to the transition between the main cylindrical portion 290 and the second cone portion 292 of the balloon 252 (see FIG. 17B). The marker bands 306, 308 can be formed of 90 percent platinum and 10 percent iridium in order to indicate by fluoroscopy, a process known in the art, the position of the balloon catheter within the patient. A soft tip 310 located distally from the balloon 252 is placed over the distal end 258 of the guidewire shaft 248.

The delivery sleeve assembly 18 is formed by joining the elongated polymeric sleeve 19 (FIGS. 9B and 9C, or 9F) and steerable section 20. The distal end 143 of the elongated polymeric sleeve 19 is inserted into the passageway 186 of the cover 148 and the passageway 154 of the flex tube 146, as shown in FIG. 10. The elongated polymeric sleeve 19 is positioned relative to the steerable section 20, such that the second tubular sleeve 140 of the elongated polymeric sleeve 19 is aligned with the curved portions 170 of the elongate openings 166 of the flex tube 146. The outer surface 144 of the elongated polymeric sleeve 19 is secured to the inner surface 150 of the flex tube 146, for example, by thermal or adhesive joining. It is understood that in the aspects where the outer jacket is present, the outer jacket forms the outer surface of the elongated polymeric sleeve. While in the aspects where the outer jacket is absent, the outer surface of the elongated polymeric sleeve is defined by the outer surface of the outer polymer layer.

Further, the barbs 164 may engage the distal end 143 of the elongated polymeric sleeve 19 to make the connection. The inner surface 184 of the cover 148 is also secured to the outer surface 144 of the elongated polymeric sleeve 19 at the proximal end 178 of the cover 148 by adhesive or thermal joining.

In the alternative aspect (see FIG. 12) involving the connector 188, the outer surface 144 of the elongated polymeric sleeve 19 is secured at its distal end 143 to the inner surface 198 of the connector 188 toward the proximal end 190 of the connector 188. The distal end 143 of the elongated polymeric sleeve 19 abuts the annularly shaped flange 196 of the connector 188.

The shroud section 21 is also joined to the steerable section 20 to form the delivery sleeve assembly 18 (see FIG. 10). The proximal end 206 of the shroud 200 is inserted into the passageway 186 of the cover 148 at the distal end 180 of the cover 148. The proximal end 206 of the shroud 200 is further inserted into the passageway 154 of the flex tube 146 at the distal end 160 of the flex tube 146. The slot 214 of the shroud 200 is aligned with the notch 176 of the flex tube 146 (see also FIGS. 11 and 14A).

The outer surface 218 of the shroud 200 in the area of the rim 204 is secured to the inner surface 150 of the flex tube 146. The proximal outer surface 226 of the ring 202 is secured to the inner surface 150 of the flex tube 146 adjacent the distal end 160 of the flex tube 146. The distal end 160 of the flex tube 146 abuts the outer face 232 of the ring 202. The shroud section 21 can be secured to the flex tube 146 with mechanical bond and adhesive.

The inner surface 184 of the cover 148 is secured to the distal outer surface 228 of the ring 202. The inner surface 184 of the cover 148 is also secured to the outer surface 218 of the shroud 200 in the area of the main body 208. These connections can be made by adhesive or thermal joining or both. The main body 208 of the shroud 200 extends distally from the distal end 180 of the cover 148.

The delivery sleeve assembly 18 is connected to the handle 22 as the proximal end 142 of the elongated polymeric sleeve 19 is inserted into the passageway 88 of the hub 30, and the outer surface 144 of the elongated polymeric sleeve 19 is secured to the inner surface of the hub 30, for example, by an adhesive.

A pull wire 312 shown in FIG. 2 is inserted into the delivery system 10. The pull wire 312 can be formed of nitinol or stainless steel. A first end of the pull wire 312 is placed in the first fastener opening 44 of the first core member 26. The first core member fastener (not shown) bears upon ball bearing 122, which secures the pull wire 312 in the first fastener opening 44. The pull wire 312 passes through the longitudinally extending access opening 46 (see FIG. 3B) of the first core member 26. The pull wire 312 passes through the passageway of the guide tube 32, which is located in the slot 40 of the first core member 26, the guide tube opening 138 of the slab 134, and the slot 82 of the second core member 29, and then through the passageway 88 of the hub 30. The pull wire 312 then passes through the second tubular sleeve 140 of the elongated polymeric sleeve 19 (see FIG. 9). The pull wire 312 exits the elongated polymeric sleeve 19 and passes through the passageway 154 of the flex tube 146 (see FIG. 10). The pull wire 312 is fixedly coupled to the ring 202 by any known mechanical and/or chemical fastener. For example, the pull wire can be coupled to the ring 202 using an adhesive, a bayonet coupling, a snap-fit, press-fit, taper fit, threaded coupling, or heat treatment (e.g., weld).

Alternatively, the pull wire 312 is movably coupled to the ring 202 (e.g., “looped through” the ring 202) and returned through the delivery system components to the first core member 26. In this example, the pull wire 312 passes through the passageway 154 of the flex tube 146 (see FIG. 10) to the ring 202. The pull wire 312 then passes through the first semi-cylindrical recess 244 and the first lumen 240 of the ring 202. The pull wire 312 is strung against the slot face 238 of the ring 202. The pull wire 312 is then returned through the second lumen 242 and the second semi-cylindrical recess 246 of the ring 202. The pull wire 312 passes again through the passageway 154 of the flex tube 146. The pull wire 312 passes through the second outer lumen 140 of the delivery elongated polymeric sleeve 19 (FIG. 9D), through the passageway 88 of the hub 30 (again), through the passageway of the guide tube 32 (again), and through the access opening 46 of the slot 40 of the first core member 26. A second end of the pull wire 312 is secured to the first core member 26 by pressure exerted by the first core member fastener (not shown) on the ball bearing 122, which secures the pull wire 312.

With reference now to FIGS. 1 and 16, a method of using the heart valve delivery system 10 will now be described in more detail. The devices and methods disclosed herein are particularly well-suited for replacing a stenotic aortic valve. Those skilled in the art will recognize that it may be necessary to pre-dilate the leaflets of the stenotic aortic valve before deploying a prosthetic valve within the aortic valve. Pre-dilation increases the flow area through the aortic valve and creates an opening in the leaflets of sufficient size to receive the prosthetic valve. Pre-dilatation can be achieved using an expandable member, such as a dilatation balloon catheter. Additional details regarding pre-dilatation and valve replacement can be found in U.S. Pat. No. 6,908,481, which is incorporated herein by reference in its whole entirety.

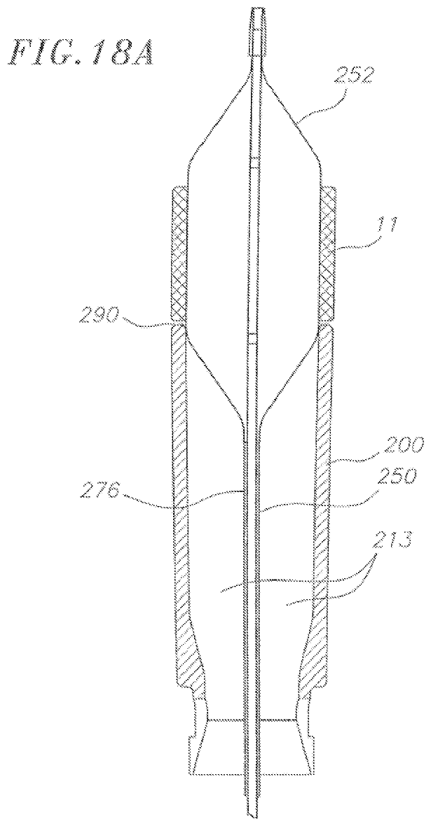

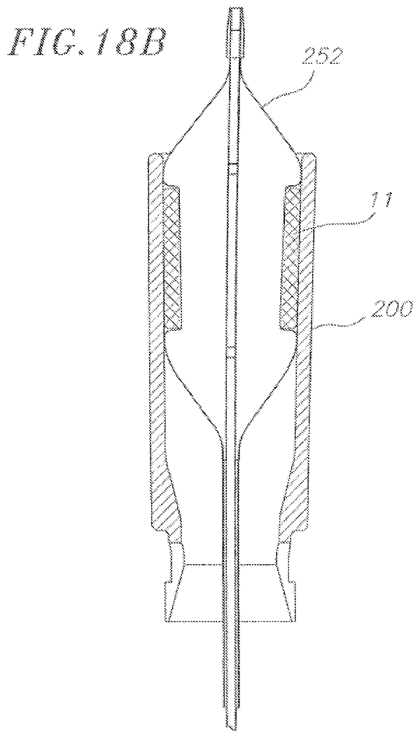

The assembly and operation of the heart valve delivery system 10 will now be described. During assembly, the balloon catheter 15 is inserted into the opening created by the assembly of the handle 22 and the delivery sleeve assembly 18. The support 17 of the balloon catheter 15 is located proximally to the handle 22. The balloon shaft 250, and the guidewire shaft 248, pass through the passageway 130 of the end cap 23 (see FIG. 2), the passageway 33 of the first core member 26, the central opening 136 of the slab 134, the passageway 78 of the second core member 29, the passageway 88 of the hub 30, the first lumen 139 of the first tubular sleeve of the elongated polymeric sleeve 19, and the passageway 154 of the flex tube 146. The balloon shaft 250 passes into the passageway 213 of the shroud 200, according to FIG. 18A, while the guidewire shaft 248 passes through the passageway 213 of the shroud 200. The proximal end 276 of the balloon 252 is located in the passageway 213 of the shroud 200, and the balloon 252 extends distally from the distal end 210 of the shroud 200.

The prosthetic valve 11 is mounted onto the main cylindrical portion 290 of the balloon 252, distally from the distal end 210 of the shroud 200, as shown in FIG. 18A. The valve 11 is known in the art and is collapsible to a first position over the balloon 252, as shown in FIG. 1. Alternatively, the valve 11 can be mounted on the balloon 252 and placed inside the shroud 200, as shown in FIG. 18B.

The valve 11 can take a variety of different forms. In some aspects, the valve generally comprises an expandable stent portion that supports a valve structure. The stent portion has sufficient radial strength to hold the valve at the treatment site and resist recoil of the stenotic valve leaflets. Additional details regarding exemplary balloon-expandable valve aspects can be found in Applicant's U.S. Pat. Nos. 6,730,118 and 6,893,460, each entitled IMPLANTABLE PROSTHETIC VALVE, which are incorporated by reference herein. It will also be appreciated that the delivery system may be used with self-expanding prosthetic valves. For example, when using a self-expanding valve, a pusher may be substituted for the balloon catheter for ejecting the self-expanding valve from the delivery sleeve assembly.

With continued reference to the illustrated aspect, the guidewire 14 is placed in the passageway 264 of the guidewire shaft 248 such that it extends distally from the distal end 258 of the guidewire shaft 248 and proximally from the wire inlet opening 296 of the support 17 of the balloon catheter 15. The process of inserting a catheter into the human body for tracking is known in the art, e.g., by U.S. Pat. No. 5,968,068 entitled ENDOVASCULAR DELIVERY SYSTEM, which is incorporated by reference herein.

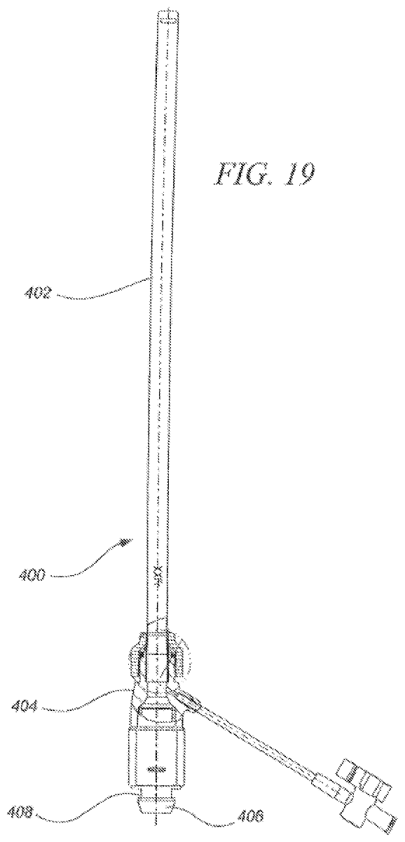

The guidewire 14 is placed in the body through a dilator (not shown), which expands the inner diameter of the body vessel in order to introduce an introducer sheath assembly 400, shown in FIG. 19, over the guidewire 14. Dilator diameters range between about 12 and about 22 French, including exemplary values of about 13, about 14, about 15, about 16, about 17, about 18, about 19, about 20, and about 21 French. The introducer sheath assembly 400 includes an introducer sleeve 402 and an introducer housing 404 attached to a proximal end of the introducer sleeve 402. Introducer sheath assembly has diameters of 22 or 24 French.

A series of valves are located inside the introducer housing 404. On a proximal end of the introducer housing 404, an end piece 406 is attached, the end piece having an opening extending into the introducer housing 404 in the area of the series of valves, and a ridge 408 facing a distal end of the introducer housing 404. The introducer sleeve 402 extends into the body vessel, with the introducer housing 404 located outside the body vessel on a proximal end on a proximal end of the introducer sleeve 402. In an aspect, the introducer sleeve 402 is coated with a hydrophilic coating and extends into the body vessel about 9 inches, just past the iliac bifurcation and into the abdominal aorta of the patient. The introducer sheath assembly 400 provides a mechanism for advancing the prosthetic valve into the aorta in a safe and effective manner.

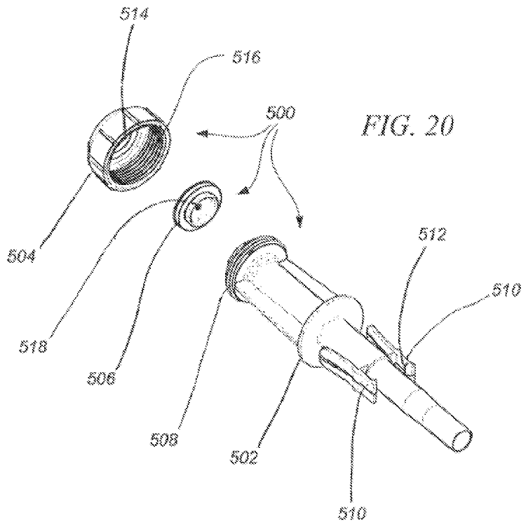

With reference to FIG. 20, a loader assembly 500 includes a loader 502, a loader cap 504, and a loader seal 506. The loader 502 is tube-shaped, having exterior threading 508 at a proximal end for connection with the loader cap 504. The loader 502 includes flexible flanges 510 extending parallel thereto and having snap ridges 512 facing the proximal end of the loader 502. The loader cap 504 includes a loader cap opening 514 in a proximal end thereof and a threaded inner surface 516 for engagement with the exterior threading 508 of the loader 502. The loader seal 506 is secured to the loader cap 504, and a loader seal opening 518 is aligned with the loader cap opening 514.

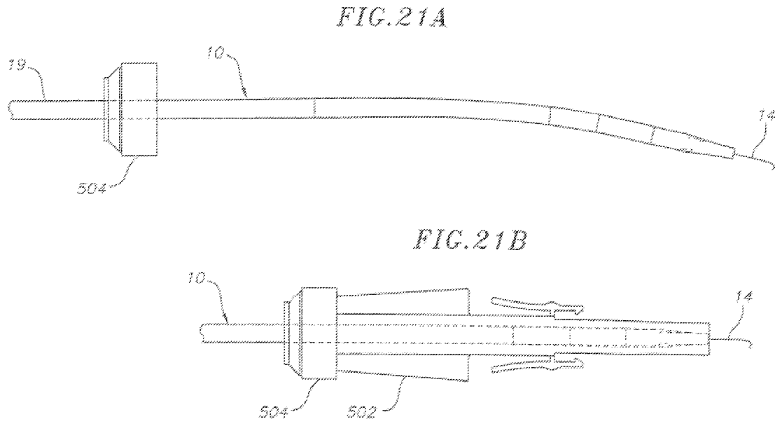

With reference to FIG. 21A, the loader cap 504 and loader seal 506 are passed onto the delivery system 10 as the elongated polymeric sleeve 19 engages the loader cap opening 514 and loader seal opening 518. The distal end of the delivery system 10, passing over the guidewire 14, is inserted into the proximal end of the loader 502, as shown in FIG. 21B. The loader cap 504 screws onto the proximal end of the loader 502.

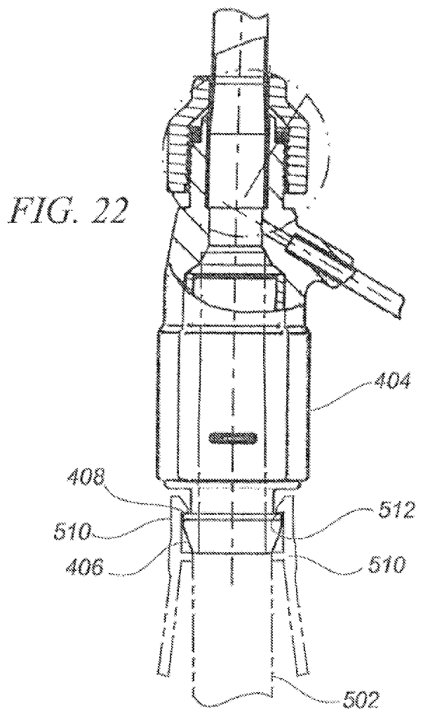

With reference to FIG. 22, the flexible flanges 510 of the loader 502 snap into the end piece 406 of the introducer housing 404. In this position, the ridge 408 of the end piece 406 bears against the snap ridge 512 of the flexible flanges 510, and the loader 502 passes through the series of valves located inside the introducer housing 404, thus placing the delivery system 10 in communication with an inner passageway of the introducer sheath and thus, with the body vessel. The loader assembly 500 advantageously allows the introduction of the delivery system 10 into the introducer sheath assembly 400 without substantial blood loss from the patient.

The prosthetic valve 11, a balloon catheter 15, and delivery sleeve assembly 18 are advanced over the guidewire 14 through the introducer sheath, as a single unit, for example, while tracking through the body vessel to the native valve site (see FIG. 1). In one advantageous feature, the delivery system 10 provides excellent pushability for facilitating advancement of the prosthetic valve 11 through the introducer sheath. In one aspect, the delivery system 10 provides sufficient pushability to push through an introducer sheath having an inner circumference that is 2 French size smaller than outer circumferences of the valve 11 or shroud 200.

As the prosthetic valve 11 reaches the aortic arch 13, as shown in FIG. 1, the steerable function of the delivery system 10, described below, is actuated for facilitating the advancement of valve 11 around the arch. More particularly, the bending of the steerable section 20 assists in steering the valve 11 and/or the distal end 210 of the shroud 200 (see FIG. 14A) away from the inner surface of the aortic arch 13. As a result, retrograde advancement of the valve 11 around the aortic arch 13 may be achieved without damaging the aorta 13 or the valve 11. In one exemplary delivery method, the valve is advanced over the aortic arch with little or no contact between the valve and the aorta.

In the illustrated aspect, the steerable function of the delivery system 10 is accomplished as the operator rotates the rotator handle 28 (see FIG. 2). As the rotator handle 28 is rotated, the threaded portion 68 acts in conjunction with the exterior thread 54 of the partially threaded member 27 (see FIG. 4A), which does not rotate. The rotator handle 28 thus moves linearly relative to the partially threaded member 27. The first core member, 26, also moves linearly relative to the partially threaded member 27 (see FIG. 2). The dowel 124 prevents relative rotation between the first core member 26 and the partially threaded member 27.

As the first core member 26 moves distally from the partially threaded member 27, the pull wire 312, connected to the first core member 26 by the ball bearing 122, exerts a force on the slot face 238 of the ring 202 (see FIG. 15A). The pull wire 312 draws the ring 202 toward handle 22. The side of the delivery system 10 along which the pull wire 312 passes bends along the steerable section 20 as the elongate openings 166 of the flex tube 146 converge (see FIG. 11). The steerable section 20 bends until the pressure in the pull wire 312 is relieved. Additional rotation of the rotator handle 28, thus results in additional bending. The friction between the threaded portion 68 of the rotator handle 28 and the exterior thread 54 of the partially threaded member 27 (see FIGS. 4A and 5B) is sufficient to hold the pull wire 312 taut, thus preserving the shape of the bend in the steerable section 20 when the operator releases the rotator handle 28.

The natural rigidity of the cover 148 (see FIG. 10), as well as the natural rigidity of the balloon catheter 15 (see FIG. 16), act against the bending of the steerable section 20. The force on the pull wire 312 bends the steerable section 20, while the rigidity of the cover 148 and balloon catheter 15 described above resists the bending, thus “locking” the delivery system 10 in place over a range of positions from straight to fully curved, according to the rotation of the rotator handle 28. The cover 148 also protects the body vessel from the flex tube 146 (see FIG. 10), which, absent the cover 148, may scrape or otherwise lacerate the body vessel.

As the balloon catheter 15 is advanced to the native valve site, the operator uses the marker bands 306, 308 (see FIG. 16) to identify the location of the valve 20, according to the process of fluoroscopy, which is well known in the art. The operator can adjust the position of the valve 11 by actuating the rotator handle 28 while holding the hub 30 stationary (see FIG. 2). Further control over valve position can be achieved by twisting the hub 30. The elongated polymeric sleeve 19 is attached to the hub 30, and the delivery system 10 is sufficiently rigid to transmit the twisting movement to the distal end. Twisting motion is transferred through the steerable section 20 when the tube portions 174 of the flex tube 146 contact one another (see FIG. 11). Such contact can occur when the flex tube is fully bent or can occur during twisting as the curved portions 170 of the elongate opening close such that the tube portions 174 contact one another.

The delivery sleeve assembly 18 (see FIG. 1) is at its most rigid when all of the remaining tube portions 174 of the flex tube 146 (see FIG. 11) are in contact with one another, and the steerable section 20 is fully curved. In this position, the shape of the steerable section 20 corresponds substantially to the shape of the aortic arch 13 (as shown in FIG. 1) for ease of tracking. When pushing across the stenotic leaflets 12, the steerable section 20 is located in the ascending aorta of the patient, and the soft durometer section of the elongated polymeric sleeve 19 flexes and bears against the aortic arch 13 (see FIG. 1), thereby preventing damage to the inner wall of the aorta.

After the delivery system 10 has been advanced, such that the valve 11 is located adjacent to the native valve, the balloon catheter 15 may be distally advanced relative to the delivery sleeve assembly 18 to better position the valve 11 within the native leaflets. To accomplish this, the balloon catheter 15 is slidably advanced through the elongated polymeric sleeve 19 (as shown in FIGS. 9C and 9F) and steerable section 20. In another advantageous feature, the delivery sleeve assembly 18 advantageously allows the physician to adjust a curvature of the steerable section 20 for properly aligning the prosthetic valve 11 with respect to the native valve. As a result, when the balloon catheter 15 is advanced distally, the prosthetic valve advances into the center of the native valve. Furthermore, the delivery system 10 provides sufficient pushability to push the balloon catheter 15 and valve 11 across the stenotic leaflets 12, or alternatively, to push the balloon catheter 15 across the stenotic leaflets 12. The shroud 200 (see FIG. 14A) may also cross the stenotic leaflets 12 during this process.

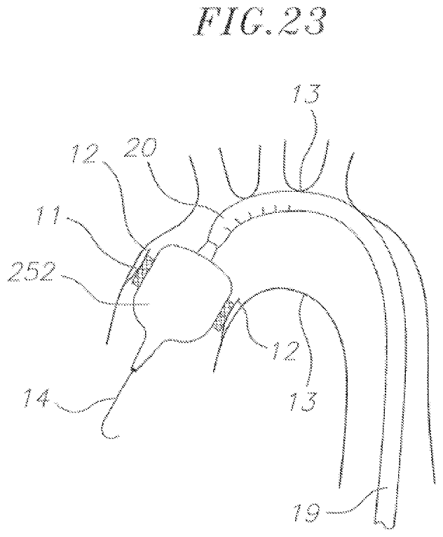

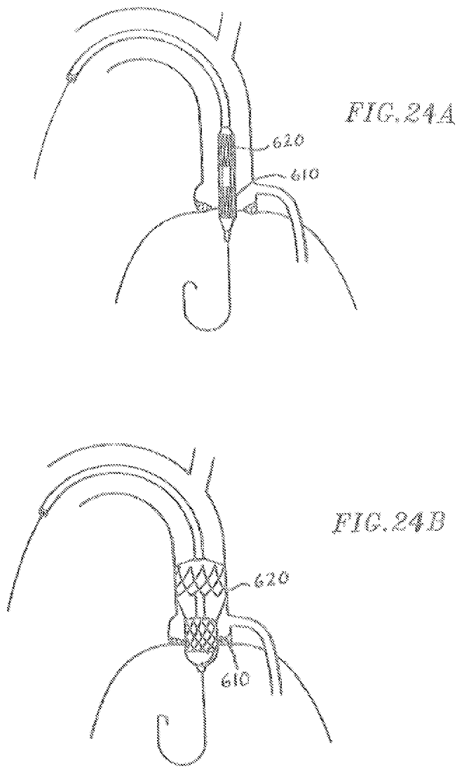

Once the stenotic leaflets 12 have been pushed away, the delivery system 10 deploys the valve 11 in the native valve site, as shown in FIG. 23. The soft durometer section of the elongated polymeric sleeve 19 (as shown, for example, in FIGS. 9C and 9F) bears against the aortic arch 13, while the steerable section 20 passes through the ascending aorta and is adjusted to position the valve 11. The valve 11 is balloon expandable, and once positioned, the balloon 252 is inflated to secure the position of the valve 11 in the native valve site. The balloon 252 is then deflated, and the entire delivery system 10 is withdrawn as it passes back over the guidewire 14 and exits the body vasculature through the introducer sheath. The guidewire 14 is then withdrawn, followed by the introducer sheath.

In the alternative aspect of the disclosure, wherein the valve 11 is placed inside the shroud 200, the delivery sleeve assembly 18 (see FIG. 1) is retracted once the valve 11 has reached the native valve site. The delivery sleeve assembly 18 is retracted as the operator holds the support 17 steady and pulls back (proximally) on the handle 22, which causes the delivery sleeve assembly 18 to retract proximally, exposing the valve 11 to the native valve site and allowing the balloon 252 to inflate as shown in FIG. 23, and thus deploy the valve 11 as described above.

It will be appreciated that aspects of the heart valve delivery system 10 provide improved devices and methods for advancing a prosthetic heart valve through a patient's vasculature. In one exemplary aspect, the cooperation of components described herein allows an uncovered prosthetic valve to be advanced through the patient's vasculature and around the aortic arch in a safe manner. Accordingly, the delivery system enables advancement of a prosthetic valve around the aortic arch without requiring the introduction of an outer sheath into the aortic arch. This is an advantageous feature because the use of a sheath would increase the diameter of the delivery system, thereby complicating the delivery of the valve. In addition to providing an improved steering mechanism for navigating the aortic arch without damaging the inner wall of the aorta, it will be appreciated by those skilled in the art that the delivery system provides excellent pushability such that the physician has excellent control over the movement and location of the prosthetic valve during advancement into the native valve. This feature is particularly advantageous when traversing stenotic valve leaflets. Accordingly, aspects of the present disclosure provide an improved delivery system for advancing a prosthetic valve, for example, to the site of a native aortic valve using a steerable assembly that eliminates the need for an outer sheath in the aorta while providing sufficiently pushability to pass through narrow vasculature and/or stenotic valve leaflets. As a result, aspects of the present disclosure provide improved devices and methods for percutaneously advancing a balloon-expandable prosthetic valve to the site of a stenotic aortic valve using a retrograde approach.

### EXEMPLARY ASPECTS

EXAMPLE 1: A delivery system for deploying a prosthetic valve comprising: an elongated polymeric sleeve having a proximal end and a distal end and comprising an inner polymer layer and an outer polymer layer; wherein the inner polymer layer forms: a first tubular sleeve having an inner surface defining a first lumen having a first diameter, an outer surface, and a first longitudinal axis; and a second tubular sleeve that is permanently adjacent to the first tubular sleeve, wherein the second tubular sleeve has an inner surface defining a second lumen having a second diameter, and an outer surface that is an extension of the outer surface of the first tubular sleeve and wherein the second tubular sleeve has a second longitudinal axis; wherein the second diameter is substantially smaller than the first diameter; and wherein the second longitudinal axis is offset of the first longitudinal axis and is substantially parallel to the first longitudinal axis along a length of the elongated polymeric sleeve; and wherein the outer surface of the first tubular sleeve and the second tubular sleeve defines an outer surface of the inner polymer layer; and wherein the inner surface of the first tubular sleeve defines an inner surface of the elongated polymeric sleeve, wherein the outer polymer layer extends circumferentially around the inner polymer layer, wherein the outer polymer layer has an inner surface and an outer surface, wherein the inner surface of the outer polymer layer is adjacent and bonded to the outer surface of the inner polymer layer; wherein the inner polymer layer comprises a fluoropolymer; and wherein the outer polymer layer comprises a polyamide, or a polyether block amide, or a combination thereof.

EXAMPLE 2: The delivery system of any examples herein, particularly example 1, wherein the inner polymer layer and the outer polymer layer of the elongated polymeric sleeve are co-extruded.

EXAMPLE 3: The delivery system of any examples herein, particularly example 1 or 2, wherein the inner polymer layer has a thickness from about 1 to about 5 mils.

EXAMPLE 4: The delivery system of any examples herein, particularly examples 1-3, wherein the outer polymer layer has a thickness from about 1 to about 5 mils.

EXAMPLE 5: The delivery system of any examples herein, particularly examples 1-4, wherein the polyamide comprises nylon 12.

EXAMPLE 6: The delivery system of any examples herein, particularly examples 1-5, wherein the elongated polymeric sleeve further comprises an outer jacket, wherein the outer jacket is disposed over the outer surface of the outer polymer layer of the elongated polymeric sleeve.

EXAMPLE 7: The delivery system of any examples herein, particularly example 6, wherein the elongated polymeric sleeve further comprises a braided layer disposed between the outer jacket and the outer surface of the outer polymer layer of the elongated polymeric sleeve.

EXAMPLE 8: The delivery system of any examples herein, particularly examples 6 or 7, wherein the inner and outer polymer layers of the elongated polymeric sleeve and/or braided layer and/or the outer jacket are fused together.

EXAMPLE 9: The delivery system of any examples herein, particularly examples 6-8, wherein the outer jacket comprises a polyether block amide.

EXAMPLE 10: The delivery system of any examples herein, particularly examples 1-9, wherein the system further comprises a selectively steerable section having a proximal end and a distal end, wherein the proximal end of the steerable section is permanently coupled to the distal end of the elongated polymeric sleeve, wherein the steerable section comprises a central lumen having an inner diameter, wherein the central lumen is coaxial with the first lumen of the first tubular sleeve, and wherein the selectively steerable section is substantially more flexible than the elongated polymeric sleeve; and wherein the selectively steerable section is configured to provide a sufficient curvature to navigate around an aortic arch.

EXAMPLE 11: The delivery system of any examples herein, particularly example 10, further comprising a pull wire having a proximal end and a distal end, wherein the distal end of the pull wire is coupled with a distal end of the steerable section, and wherein the pull wire is configured to selectively control a curvature of the steerable section.

EXAMPLE 12: The delivery system of any examples herein, particularly example 11, wherein the pull wire is extending through the second lumen of the second tubular sleeve and is configured to pass through the steerable section to the distal end of the steerable section.

EXAMPLE 13: The delivery system of any examples herein, particularly examples 10-12, further comprising an elongate balloon catheter extending through the first tubular sleeve of the elongated polymeric sleeve and through the central lumen of the steerable section and out of the distal end of the steerable section, and wherein the elongate balloon catheter being longitudinally movable relative to the first tubular sleeve and the steerable section.

EXAMPLE 14: The delivery system of any examples herein, particularly example 13, further comprising a prosthetic valve disposed over an expandable balloon along a distal end of the elongate balloon catheter, wherein the balloon has a deflated position and an inflated position; wherein the prosthetic valve has an outer diameter larger than the inner diameter of the central lumen of the steerable section such that the prosthetic valve is prevented from moving proximally through the central lumen of the steerable section when the balloon is in the deflated position and wherein the prosthetic valve is in a radially compressed state on the deflated balloon, and wherein the prosthetic valve comprises an expandable stent portion and a valve structure.

EXAMPLE 15: The delivery system of any examples herein, particularly example 14, wherein the elongated polymeric sleeve, steerable section, balloon catheter, and prosthetic valve are configured for advancement as a single unit through a patient's vasculature while the prosthetic valve is positioned distal to the steerable section and wherein the balloon catheter and prosthetic valve are movable in a distal direction relative to the elongated polymeric sleeve and the steerable section for implanting the prosthetic valve within a native aortic valve.

EXAMPLE 16: The delivery system of any examples herein, particularly examples 10-15, wherein the steerable section comprises a slotted tube having a first straight position and a second curved position.

EXAMPLE 17: The delivery system any one of any examples herein, particularly examples 10-16, wherein the steerable section further comprises a flexible tubular portion and an exterior cover extending over at least a portion of the tubular portion, the cover being less flexible than the tubular portion.

EXAMPLE 18: The delivery system of any examples herein, particularly example 17, wherein the flexible tubular portion is a metal tubular portion comprising a plurality of axially spaced-apart, circumferentially extending openings.

EXAMPLE 19: The delivery system of any examples herein, particularly example 18, wherein the metal tubular portion comprises a stainless-steel hypo tube.

EXAMPLE 20: The delivery system of any examples herein, particularly example 18 or 19, wherein each opening in the tubular portion of the steerable section comprises two elongate portions connected by a curved portion.

EXAMPLE 21: The delivery system of any examples herein, particularly examples 17-20, wherein the exterior cover is polymeric and extends over the openings in the tubular portion.

EXAMPLE 22: The delivery system of any examples herein, particularly examples 20-21, wherein the second lumen of the second sleeve is circumferentially aligned with the curved portion of the openings of the steerable portion.

EXAMPLE 23: The delivery system of any examples herein, particularly examples 10-22, wherein the outer polymer layer near the distal end of the elongated polymeric sleeve comprises a durometer different from the outer polymer layer near the proximal end of the elongated polymeric sleeve.

EXAMPLE 24: The delivery system of any examples herein, particularly examples 10-23, wherein the system further comprises a handle coupled to the proximal end of the elongated polymeric sleeve.

EXAMPLE 25: The delivery system of any examples herein, particularly example 24, wherein the handle is coupled to the proximal end of the pull wire.

EXAMPLE 26: The delivery system of any examples herein, particularly examples 24-25, wherein the handle comprises a steering mechanism for actuating the pull wire to selectively control a curvature of the steerable section during advancement of the delivery.

EXAMPLE 27: The delivery system of any examples herein, particularly example 26, wherein the steering mechanism is effective to move the pull wire such that the elongated polymeric sleeve exhibits substantially no deflection during a movement of the pull wire.

EXAMPLE 28: The delivery system of any examples herein, particularly examples 10-27, wherein the distal end of the steerable section abuts a proximal end of the prosthetic valve.

EXAMPLE 29: The delivery system of any examples herein, particularly examples 14-28, further comprising a shroud coupled to the distal end of the steerable section and wherein the shroud surrounds at least a portion of the prosthetic valve during advancement through the patient's vasculature.

EXAMPLE 30: An assembly comprising: a self-expanding prosthetic valve comprising a radially compressible and expandable metallic stent and a flexible valvular structure mounted within the stent; and a delivery apparatus comprising a delivery sleeve assembly comprising: an elongated polymeric sleeve having a proximal end and a distal end and comprising an inner polymer layer and an outer polymer layer; wherein the inner polymer layer forms: a first tubular sleeve having an inner surface defining a first lumen having a first diameter, an outer surface, and a first longitudinal axis; and a second tubular sleeve that is permanently adjacent to the first tubular sleeve, wherein the second tubular sleeve has an inner surface defining a second lumen having a second diameter, and an outer surface that is an extension of the outer surface of the first tubular sleeve and wherein the second tubular sleeve has a second longitudinal axis; wherein the second diameter is substantially smaller than the first diameter; and wherein the second longitudinal axis is offset of the first longitudinal axis and is substantially parallel to the first longitudinal axis along a length of the elongated polymeric sleeve; and wherein the outer surface of the first tubular sleeve and the second tubular sleeve defines an outer surface of the inner polymer layer; and wherein the inner surface of the first tubular sleeve defines an inner surface of the elongated polymeric sleeve, wherein the outer polymer layer extends circumferentially around the inner polymer layer, wherein the outer polymer layer has an inner surface and an outer surface, wherein the inner surface of the outer polymer layer is adjacent and bonded to the outer surface of the inner polymer layer; wherein the inner polymer layer comprises a fluoropolymer; and wherein the outer polymer layer comprises a polyamide, or a polyether block amide, or a combination thereof.

EXAMPLE 31: The assembly of any examples herein, particularly example 30, wherein the inner polymer layer and the outer polymer layer of the elongated polymeric sleeve are co-extruded.

EXAMPLE 32: The assembly of any examples herein, particularly example 30 or 31, wherein the inner polymer layer has a thickness from about 1 to about 5 mils.

EXAMPLE 33: The assembly of any examples herein, particularly examples 30-32, wherein the outer polymer layer has a thickness from about 1 to about 5 mils.

EXAMPLE 34: The assembly of any examples herein, particularly examples 30-33, wherein the polyamide comprises nylon 12.

EXAMPLE 35: The assembly of any one claim 30-34, wherein the elongated polymeric sleeve further comprises an outer jacket, wherein the outer jacket is disposed over the outer surface of the outer polymer layer of the elongated polymeric sleeve.

EXAMPLE 36: The assembly of any examples herein, particularly example 35, wherein the elongated polymeric sleeve further comprises a braided layer disposed between the outer jacket and the outer surface of the outer polymer layer of the elongated polymeric sleeve.

EXAMPLE 37: The assembly of any examples herein, particularly examples 35 or 36, wherein the inner polymer layer and outer polymer layer of the elongated polymeric sleeve and/or braided layer and/or the outer jacket are fused together.

EXAMPLE 38: The assembly of any examples herein, particularly examples 36-37, wherein the outer jacket comprises a polyether block amide.

EXAMPLE 39: The assembly of any examples herein, particularly examples 30-38, wherein the delivery sleeve assembly further comprises a selectively steerable section having a proximal end and a distal end, wherein the proximal end of the steerable section is permanently coupled to the distal end of the elongated polymeric sleeve, wherein the steerable section comprises a central lumen having an inner diameter, wherein the central lumen is coaxial with the first lumen of the first tubular sleeve, and wherein the selectively steerable section is substantially more flexible than the elongated polymeric sleeve; and wherein the selectively steerable section is configured to provide a sufficient curvature to navigate around an aortic arch.

EXAMPLE 40: The assembly of any examples herein, particularly example 39, wherein the prosthetic valve is retained in a radially compressed state within an open distal end of the delivery sleeve assembly.

EXAMPLE 41: The assembly of any examples herein, particularly example 40, wherein the delivery sleeve assembly further comprises a pusher member extending through the central lumen of the steerable section, the pusher member having a distal end located proximal to the prosthetic valve.

EXAMPLE 42: The assembly of any examples herein, particularly examples 39-41, further comprising a pull wire having a proximal end and a distal end, wherein the distal end of the pull wire is coupled with the distal end of the steerable section, wherein the pull wire is configured to selectively control a curvature of the steerable section.

EXAMPLE 43: The assembly of any examples herein, particularly example 42, wherein the pull wire is extending through the second lumen of the second tubular sleeve and configured to pass through the steerable section to the distal end of the steerable section.

EXAMPLE 44: The assembly of any examples herein, particularly examples 39-43, wherein the steerable section comprises a slotted tube having a first straight position and a second curved position.

EXAMPLE 45: The assembly of any one of examples herein, particularly examples 39-44, wherein the steerable section further comprises a flexible tubular portion and an exterior cover extending over at least a portion of the tubular portion, the cover being less flexible than the tubular portion.

EXAMPLE 46: The assembly of any examples herein, particularly example 45, wherein the flexible tubular portion is a metal tubular portion comprising a plurality of axially spaced-apart, circumferentially extending openings.

EXAMPLE 47: The assembly of any examples herein, particularly example 46, wherein the metal tubular portion comprises a stainless-steel hypo tube.

EXAMPLE 48: The assembly of any examples herein, particularly example 46 or 47, wherein each opening in the tubular portion of the steerable section comprises two elongate portions connected by a curved portion.

EXAMPLE 49: The assembly of any examples herein, particularly examples 45-48, wherein the exterior cover is polymeric and extends over the openings in the tubular portion.

EXAMPLE 50: The assembly of any examples herein, particularly examples 48-49, wherein the second lumen of the second sleeve is circumferentially aligned with the curved portion of the openings of the steerable portion.

EXAMPLE 51: The assembly of any examples herein, particularly examples 30-50, wherein the outer polymer layer near the distal end of the elongated polymeric sleeve comprises a durometer different from the outer polymer layer near the proximal end of the elongated polymeric sleeve.

EXAMPLE 52: The assembly of any examples herein, particularly examples 30-51, wherein the delivery sleeve assembly further comprises a handle coupled to a proximal end of the polymeric tubing.

EXAMPLE 53: The assembly system of any examples herein, particularly example 52, wherein the handle comprises a steering mechanism for actuating the pull wire to selectively control a curvature of the steerable section during advancement of the delivery.

EXAMPLE 54: The assembly of any examples herein, particularly example 53, wherein the steering mechanism is effective to move the pull wire such that the polymer tubing exhibits substantially no deflection during a movement of the pull wire.

EXAMPLE 55: The assembly of any examples herein, particularly examples 41-54, wherein the delivery sleeve assembly is configured to be retracted relative to the pusher member for ejecting the prosthetic valve from the system and into a native aortic valve, wherein the prosthetic valve is configured to self-expand after ejection from the delivery sleeve assembly.

EXAMPLE 56: The assembly of any examples herein, particularly examples 41-55, wherein the distal end of the pusher member is configured to abut a proximal end of the prosthetic valve for pushing the prosthetic valve out of the delivery system.

EXAMPLE 57: The assembly of any examples herein, particularly examples 42-56, wherein the distal end of the pull wire is fixed to the steerable section at a location proximal to the prosthetic valve retained within the distal end of the delivery sleeve assembly.

EXAMPLE 58: The assembly of any examples herein, particularly example 57, wherein the steerable section is substantially more flexible than the distal end of the delivery sleeve assembly such that the distal end of the delivery sleeve assembly remains straight when the pull wire is actuated to change a curvature of the steerable section so that the prosthetic valve retained within the distal end of the delivery sleeve assembly can be aligned with the native aortic valve.

EXAMPLE 59: A method of making a delivery sleeve assembly comprising: forming an elongated polymeric sleeve having a proximal end and a distal end and comprising an inner polymer layer and an outer polymer layer; wherein the inner polymer layer forms: a first tubular sleeve having an inner surface defining a first lumen having a first diameter, an outer surface, and a first longitudinal axis; and a second tubular sleeve that is permanently adjacent to the first tubular sleeve, wherein the second tubular sleeve has an inner surface defining a second lumen having a second diameter, and an outer surface that is an extension of the outer surface of the first tubular sleeve and wherein the second tubular sleeve has a second longitudinal axis; wherein the second diameter is substantially smaller than the first diameter; and wherein the second longitudinal axis is offset of the first longitudinal axis and is substantially parallel to the first longitudinal axis along a length of the elongated polymeric sleeve; and wherein the outer surface of the first tubular sleeve and the second tubular sleeve defines an outer surface of the inner polymer layer; and wherein the inner surface of the first tubular sleeve defines an inner surface of the elongated polymeric sleeve, wherein the outer polymer layer extends circumferentially around the inner polymer layer, wherein the outer polymer layer has an inner surface and an outer surface, wherein the inner surface of the outer polymer layer is adjacent and bonded to the outer surface of the inner polymer layer.

EXAMPLE 60: The method of any examples herein, particularly example 59, wherein the step of forming the elongated polymeric sleeve comprises co-extruding a first polymer to form the inner polymer layer and a second polymer to form the outer polymer layer.

EXAMPLE 61: The method of any examples herein, particularly example 60, wherein the first polymer comprises a fluoropolymer.

EXAMPLE 62: The method of any examples herein, particularly examples 60 or 61, wherein the second polymer comprises a polyamide, a polyether block amide, or a combination thereof.

EXAMPLE 63: The method of any examples herein, particularly example 62, wherein the polyamide comprises nylon 12.

EXAMPLE 64: The method of any examples herein, particularly examples 59-63, wherein the inner polymer layer has a thickness from about 1 to about 5 mils.

EXAMPLE 65: The method of any examples herein, particularly examples 59-64, wherein the outer polymer layer has a thickness from about 1 to about 5 mils.

EXAMPLE 66: The method of any examples herein, particularly examples 59-65, wherein the outer polymer layer near the distal end of the elongated polymeric sleeve comprises a durometer different from the outer polymer layer near the proximal end of the elongated polymeric sleeve.

EXAMPLE 67: The method of any examples herein, particularly examples 59-66, wherein the step of forming the elongated polymeric sleeve further comprises disposing an outer jacket on the outer surface of the outer polymer layer such that the inner polymer layer and the outer polymer layer are enclosed within the outer jacket.

EXAMPLE 68: The method of any examples herein, particularly example 67, further comprising a step of providing a braided layer over the outer surface of the outer polymer layer prior to the step of disposing the outer jacket.

EXAMPLE 69: The method of any examples herein, particularly example 67 or 68, wherein the outer jacket comprises a polyether block amide.

EXAMPLE 70: The method of any examples herein, particularly examples 68-69, wherein the braided layer comprises a metal mesh or a laser cut hypo tube.

EXAMPLE 71: The method of any examples herein, particularly examples 68-70, wherein the method comprises a step of heat treatment to fuse the inner polymer layer, the outer polymer layer, the braided layer, and the outer jacket together.

EXAMPLE 72: The method of any examples herein, particularly examples 59-71, further comprising forming a selectively steerable section having a proximal end and a distal end, wherein the proximal end of the steerable section is permanently coupled to the distal end of the elongated polymeric sleeve, wherein the steerable section comprises a central lumen having an inner diameter, wherein the central lumen is coaxial with the first lumen of the first tubular sleeve, and wherein the selectively steerable section is substantially more flexible than the elongated polymeric sleeve; and wherein the selectively steerable section is configured to provide a sufficient curvature to navigate around an aortic arch.

EXAMPLE 73: The method of any examples herein, particularly example 72, wherein the steerable section comprises a slotted tube having a first straight position and a second curved position.

EXAMPLE 74: The method of any examples herein, particularly example 72 or 73, wherein the steerable section comprises a flexible metal tubular portion comprising a plurality of axially spaced-apart, circumferentially extending openings and an exterior polymeric cover extending over an entire outer surface of the tubular portion and covering the openings in the tubular portion, the cover being less flexible than the tubular portion.

EXAMPLE 75: The method of any examples herein, particularly examples 72-74, wherein at least a portion of the steerable section is formed from with a material having a soft durometer.

EXAMPLE 76: The method of any examples herein, particularly examples 74-75, wherein each opening in the tubular portion comprises two elongate straight portions connected by a curved portion.

EXAMPLE 77: The method of any examples herein, particularly example 76, wherein the second lumen of the second sleeve is circumferentially aligned with the curved portion of the openings of the steerable portion.

EXAMPLE 78: The method of any examples herein, particularly examples 72-77, further comprising coupling a distal end of a pull wire with the distal end of the steerable section, wherein the pull wire is configured to selectively control a curvature of the steerable section.

EXAMPLE 79: The method of any examples herein, particularly example 78, wherein the pull wire extends through the second lumen of the second tubular sleeve and configured to pass through the steerable section to the distal end of the steerable section.

EXAMPLE 80: The method of any examples herein, particularly examples 59-79, further comprising coupling a handle to a proximal end of the elongated polymeric sleeve.

EXAMPLE 81: The method system of any examples herein, particularly example 80, wherein the handle comprises a steering mechanism for actuating the pull wire to selectively control a curvature of the steerable section during advancement of the delivery.

EXAMPLE 82: The method of any examples herein, particularly example 81, wherein the steering mechanism is effective to move the pull wire such that the polymer tubing exhibits substantially no deflection during a movement of the pull wire.

EXAMPLE 83: A method of deploying a prosthetic valve within a native aortic valve in a human heart, the prosthetic valve comprising a radially compressible and expandable metallic stent and a flexible valvular structure mounted within the stent, the method comprising: providing a delivery system of any examples herein, particularly examples 14-29; crimping the prosthetic valve along the distal end portion of the balloon catheter such that the prosthetic valve is positioned distal to the distal end of the steerable section; advancing the prosthetic valve, balloon catheter, elongated polymeric sleeve, and steerable section through a femoral artery and an aortic arch; actuating the pull wire for selectively controlling a curvature of the steerable section during advancement around the aortic arch; distally advancing the balloon catheter and prosthetic valve relative to the elongated polymeric sleeve and the steerable section for positioning the prosthetic valve within the native aortic valve; and radially expanding the prosthetic valve by inflating a balloon disposed along the distal end portion of the balloon catheter.

EXAMPLE 84: The method of any examples herein, particularly example 83, wherein at least a portion of the steerable section is formed with a material having a soft durometer for facilitating flexing of the steerable section.

EXAMPLE 85: The method of any examples herein, particularly example 83 or 84, wherein at least a portion of the steerable section is formed by laser cutting a stainless-steel hypo-tube.

EXAMPLE 86: The method of any examples herein, particularly examples 83-85, wherein the pull wire is actuated by a rotatable handle assembly and wherein the rotatable handle assembly is located proximal to the elongated polymeric sleeve.

EXAMPLE 87: The method of any examples herein, particularly examples 83-86, wherein the distal end of the steerable section abuts a proximal end of the prosthetic valve for pushing the prosthetic valve during advancement of the prosthetic valve through the femoral artery and the aortic arch.

EXAMPLE 88: The method of any examples herein, particularly examples 83-87, further comprising a shroud coupled to the distal end of the steerable section and wherein the shroud surrounds at least a portion of the prosthetic valve during advancement of the prosthetic valve through the femoral artery and the aortic arch.

EXAMPLE 89: The method any one of any examples herein, particularly examples 83-88, wherein the proximal end of the pull wire connected to a handle mechanism and wherein actuating the handle mechanism pulls the pull wire proximally to bend the steerable section.

EXAMPLE 90: A method of deploying a prosthetic valve within a stenotic native aortic valve, the prosthetic valve comprising a radially compressible and expandable metallic stent and a flexible valvular structure mounted within the stent, the method comprising: providing a delivery system of any examples herein, particularly examples 14-29; crimping the prosthetic valve along the distal end of the balloon catheter such that the prosthetic valve is positioned distal to the distal end of the steerable section; pre-dilating leaflets of the native aortic valve for increasing the flow area through the native aortic valve; advancing the prosthetic valve, balloon catheter, elongated polymeric sleeve, and steerable section through a femoral artery and an aortic arch; actuating the pull wire for selectively controlling a curvature of the steerable section during advancement around the aortic arch; advancing the balloon catheter and prosthetic valve relative to the elongated polymeric sleeve and the steerable section for positioning the prosthetic valve within the native aortic valve; and radially expanding the prosthetic valve by inflating a balloon disposed along the distal end portion of the balloon catheter.

EXAMPLE 91: The method of any examples herein, particularly example 90, wherein pre-dilating the leaflets of the native aortic valve is performed by expanding an expandable balloon within the native aortic valve.

EXAMPLE 92: A method of deploying a self-expanding prosthetic valve within a stenotic native aortic valve, the prosthetic valve comprising a radially compressible and expandable metallic stent and a flexible valvular structure mounted within the stent, the method comprising: providing a delivery sleeve assembly of any examples herein, particularly examples 42-58; crimping the prosthetic valve; inserting the prosthetic valve into the distal end of the delivery sleeve assembly such that the prosthetic valve is located distal to the pusher member; pre-dilating leaflets of the native aortic valve for increasing the flow area through the native aortic valve; advancing the distal end of the delivery sleeve assembly through a femoral artery and aorta; actuating the pull wire for selectively controlling a curvature of the selectively steerable section during further advancement around an aortic arch; positioning the prosthetic valve adjacent to the native aortic valve; and retracting the delivery sleeve assembly relative to the pusher member for ejecting the prosthetic valve from the delivery sleeve assembly and into the native aortic valve, wherein the prosthetic valve self-expands after ejection from the delivery sleeve assembly.

EXAMPLE 93: The method of any examples herein, particularly example 92, wherein pre-dilating the leaflets of the native aortic valve is performed by expanding an expandable balloon within the native aortic valve.

EXAMPLE 94: The method of any examples herein, particularly example 92 or 93, wherein positioning the prosthetic valve adjacent to the native aortic valve comprises pushing the delivery sleeve assembly in a retrograde direction such that a portion of the delivery sleeve assembly proximal to the prosthetic valve bears against the aortic arch and such that the prosthetic valve crosses the native aortic valve.

EXAMPLE 95: The method of any examples herein, particularly example 94, further comprising adjusting a curvature of the steerable section by actuation of the pull wire to align the prosthetic valve with respect to the center of the native valve prior to advancing the prosthetic valve across the native valve.

EXAMPLE 96: A method of deploying a self-expanding prosthetic valve in a native aortic valve without surgery, the prosthetic valve comprising a radially compressible and expandable metallic stent and a flexible valvular structure formed of pericardial tissue and sutured to the stent, the method comprising: providing a delivery sleeve assembly of any examples herein, particularly examples 42-58; crimping the prosthetic valve; inserting the prosthetic valve into the delivery sleeve assembly such that the prosthetic valve is located distal to the pusher member; advancing the prosthetic valve, pusher, elongated polymeric sleeve, and steerable section through a femoral artery and an aorta; actuating the pull wire for selectively controlling a curvature of the selectively steerable section of the delivery sleeve assembly during advancement around an aortic arch; advancing the pusher member and prosthetic valve relative to the delivery sleeve assembly for ejecting the prosthetic valve within the native aortic valve; and allowing the prosthetic valve to self-expand expand, wherein the stent of the prosthetic valve has a first portion configured to engage leaflets of the native aortic valve and a second portion configured to engage an inner wall of an ascending aorta and wherein the first portion has a smaller diameter than the second portion.

EXAMPLE 97: The method of any examples herein, particularly example 96, further comprising advancing the prosthetic valve in a retrograde direction through the aortic arch such that a portion of the delivery sleeve proximal to the prosthetic valve bears against an inner surface of the aortic arc while actuating the pull wire to steer the prosthetic valve away from the inner surface of the aortic toward the center of the native aortic valve.

EXAMPLE 98: The method of any examples herein, particularly example 97, further comprising advancing the prosthetic valve until the first portion of the stent crosses the native aortic valve.

EXAMPLE 99: A method comprising: inserting an introducer into a blood vessel of a patient, the introducer having a proximal opening, a distal opening, and an inner passageway between the proximal opening and the distal opening; inserting the delivery system of any examples herein, particularly examples 14-29 into a loader, the prosthetic valve being crimped over an inflatable balloon along a distal end of the delivery system; inserting the loader through the proximal opening of the introducer after inserting the delivery system and the prosthetic valve into the loader; and advancing the prosthetic valve and the delivery system outwardly through a distal opening of the loader into the inner passageway of the introducer and then outwardly through the distal opening of the introducer into the blood vessel; wherein the act of inserting the delivery system and the prosthetic valve into the loader comprises inserting a distal end portion of the delivery system and the crimped prosthetic valve into a tube portion of the loader; inserting the delivery system and the prosthetic valve through a cap opening of a cap portion of the loader; and inserting the delivery system and the prosthetic valve through a seal opening of a seal member, wherein the seal opening is configured to align with the cap opening and is sized such that when the delivery system is inserted through the cap opening and the seal opening, an outer surface of the delivery system engages the seal opening to block blood flow through the seal member.

EXAMPLE 100: The method of any examples herein, particularly example 99, wherein the inner passageway of the introducer has a circumference that is smaller than an outer circumference of the prosthetic valve in the radially compressed state prior to being inserted into the introducer.

EXAMPLE 101: The method of any examples herein, particularly example 99 or 100, further comprising inserting a guidewire into the blood vessel.

EXAMPLE 102: The method of any examples herein, particularly example 101, further comprising inserting a dilator over the guidewire into the blood vessel and expanding an inner diameter of the blood vessel to allow insertion of the introducer.

EXAMPLE 103: The method of any examples herein, particularly example 102, wherein the act of inserting the introducer into the blood vessel comprises advancing the introducer over the guidewire into the blood vessel.

EXAMPLE 104: The method of any examples herein, particularly example 103, wherein the act of inserting the loader through the proximal opening of the introducer comprises inserting the tube portion of the loader through one or more valves located inside the introducer, the one or more valves being configured to control blood flow inside the inner passageway.

EXAMPLE 105: The method of any examples herein, particularly examples 99-104, further comprising coupling the tube portion of the loader to the introducer.

EXAMPLE 106: The method of any examples herein, particularly examples 99-105, further comprising expanding the prosthetic valve by inflating the inflatable balloon after the prosthetic valve is delivered through the blood vessel and deployed within a native aortic valve.

EXAMPLE 107: A method comprising: inserting an introducer into a blood vessel of a patient, the introducer having a proximal opening, a distal opening, and an inner passageway between the proximal opening and the distal opening; inserting a delivery system of any examples herein, particularly examples 14-29 into a loader, the prosthetic valve being crimped over an inflatable balloon along a distal end of the delivery system; inserting the loader through the proximal opening of the introducer after inserting the delivery system and the prosthetic valve into the loader; and advancing the prosthetic valve and the delivery system outwardly through a distal opening of the loader into the inner passageway of the introducer and then outwardly through the distal opening of the introducer into the blood vessel; wherein the act of inserting the delivery system and the prosthetic valve into the loader comprises inserting a distal end portion of the delivery system and the crimped prosthetic valve into a tube portion of the loader; inserting the delivery system and the prosthetic valve through a cap opening of a cap portion of the loader and through a seal member; and coupling the cap portion to the tube portion.

EXAMPLE 108: A method comprising: inserting a sleeve of an introducer into a blood vessel of a patient so that an inner passageway of the sleeve is in fluid communication with the blood vessel, a housing of the introducer attached to a proximal end of the sleeve being located outside the blood vessel; inserting a delivery system of any examples herein, particularly examples 14-29 through a cap portion and a seal member of a loader, the prosthetic valve being crimped over an inflatable balloon of the delivery system, and the seal member being configured to engage an outer surface of the delivery system to block blood flow through the seal member; inserting a tube portion of the loader through a proximal opening of the housing and one or more valves located inside the housing, the one or more valves being configured to block blood flow from the inner passageway of the sleeve to the proximal opening of the housing, wherein an inner lumen of the tube portion contains a distal end portion of the delivery system and the prosthetic valve retained thereon; and advancing the prosthetic valve and the delivery system outwardly through a distal opening of the tube portion of the loader into the inner passageway of the sleeve and then outwardly through a distal opening of the sleeve into the blood vessel without contacting the one or more valves.

EXAMPLE 109: The method of any examples herein, particularly example 108, further comprising coupling the tube portion of the loader to the housing of the introducer.

EXAMPLE 110: The method of any examples herein, particularly example 108 or 109, further comprising inserting the distal end of the delivery system and the prosthetic valve retained thereon in the radially compressed state into the tube portion of the loader.

EXAMPLE 111: The method of any examples herein, particularly examples 108-110, wherein the vessel is a femoral artery of the patient and the method further comprises advancing the distal end portion of the delivery system and the prosthetic valve through an aorta toward a native aortic valve.

EXAMPLE 112: The method of any examples herein, particularly examples 108-111, further comprising advancing the distal end of the delivery system and the prosthetic valve through an aortic arch and actuating a steering mechanism of the delivery system to increase a curvature of the distal end portion of the delivery system.

EXAMPLE 113: The method of any examples herein, particularly example 112, further comprising positioning the prosthetic valve within the native aortic valve and radially expanding the prosthetic valve into engagement with surrounding tissue.

EXAMPLE 114: The method of any examples herein, particularly example 113, wherein radially expanding the prosthetic valve comprises inflating the inflatable balloon.

EXAMPLE 115: A method comprising: inserting a guidewire into a blood vessel of a patient; inserting a sleeve of an introducer over the guidewire into the blood vessel so that an inner passageway of the sleeve is in fluid communication with the blood vessel, a housing of the introducer attached to a proximal end of the sleeve being located outside the blood vessel; inserting a delivery system of any examples herein, particularly examples 14-29 through a cap opening of a cap portion of a loader and a seal opening of a seal member, the prosthetic valve being crimped over an inflatable balloon of the delivery system, the seal opening being configured to align with the cap opening and is sized such that when the delivery system is inserted through the cap opening and the seal opening, an outer surface of the delivery system engages the seal opening to block blood flow through the seal member; inserting the distal end portion of the delivery system and the prosthetic valve into a tube portion of the loader; coupling the cap portion to the tube portion; inserting the tube portion of the loader through a proximal opening of the housing and one or more valves located inside the housing, the one or more valves being configured to block blood flow from the inner passageway of the sleeve to the proximal opening of the housing; coupling the tube portion of the loader to the introducer; advancing the prosthetic valve and the delivery system outwardly as a single unit over the guidewire through a distal opening of the tube portion of the loader into the inner passageway of the sleeve and then outwardly through a distal opening of the sleeve into the blood vessel without contacting the one or more valves; and expanding the prosthetic valve by inflating the inflatable balloon after the prosthetic valve is delivered through the blood vessel and deployed within a native aortic valve.

EXAMPLE 116: A delivery system for deploying a prosthetic valve comprising: an elongated polymeric sleeve having a proximal end and a distal end and comprising an inner polymer layer and an outer polymer layer; wherein the inner polymer layer forms: a first tubular sleeve having an inner surface defining a first lumen having a first diameter, an outer surface, and a first longitudinal axis; and a second tubular sleeve that is permanently adjacent to the first tubular sleeve, wherein the second tubular sleeve has an inner surface defining a second lumen having a second diameter, and an outer surface that is an extension of the outer surface of the first tubular sleeve and wherein the second tubular sleeve has a second longitudinal axis; wherein the second diameter is substantially smaller than the first diameter; and wherein the second longitudinal axis is offset of the first longitudinal axis and is substantially parallel to the first longitudinal axis along a length of the elongated polymeric sleeve; and wherein the outer surface of the first tubular sleeve and the second tubular sleeve defines an outer surface of the inner polymer layer; and wherein the inner surface of the first tubular sleeve defines an inner surface of the elongated polymeric sleeve, wherein the outer polymer layer extends circumferentially around the inner polymer layer, wherein the outer polymer layer has an inner surface and an outer surface, wherein the inner surface of the outer polymer layer is adjacent and bonded to the outer surface of the inner polymer layer; wherein the inner polymer layer comprises a fluoropolymer; and wherein the outer polymer layer comprises a polyamide, or a polyether block amide, or a combination thereof.

EXAMPLE 117: The delivery system of any examples herein, particularly example 116, wherein the inner polymer layer and the outer polymer layer of the elongated polymeric sleeve are co-extruded.

EXAMPLE 118: The delivery system of any examples herein, particularly examples 116-117, wherein the fluoropolymer comprises an ethylene-perfluoroethylenepropene copolymer.

EXAMPLE 119: The delivery system of any examples herein, particularly examples 116-118, wherein the outer polymer layer is the polyamide comprising nylon 12.

EXAMPLE 120: The delivery system of any examples herein, particularly examples 116-119, wherein the elongated polymeric sleeve further comprises an outer jacket, wherein the outer jacket is disposed outward of the outer surface of the outer polymer layer of the elongated polymeric sleeve.

EXAMPLE 121: The delivery system of any examples herein, particularly example 120, wherein the elongated polymeric sleeve further comprises a braided layer disposed between the outer jacket and the outer surface of the outer polymer layer of the elongated polymeric sleeve.

EXAMPLE 122: The delivery system of any examples herein, particularly example 121, wherein the inner and outer polymer layers of the elongated polymeric sleeve and/or braided layer and/or the outer jacket are fused together.

EXAMPLE 123: The delivery system of any examples herein, particularly examples 120-122, wherein the outer jacket comprises a polyether block amide.

EXAMPLE 124: The delivery system of any examples herein, particularly examples 116-123, wherein the system further comprises a selectively steerable section having a proximal end and a distal end, wherein the proximal end of the steerable section is permanently coupled to the distal end of the elongated polymeric sleeve, wherein the steerable section comprises a central lumen having an inner diameter, wherein the central lumen is coaxial with the first lumen of the first tubular sleeve, and wherein the selectively steerable section is substantially more flexible than the elongated polymeric sleeve; and wherein the selectively steerable section is configured to provide a sufficient curvature to navigate around an aortic arch.

EXAMPLE 125: The delivery system of any examples herein, particularly example 124, further comprising a pull wire having a proximal end and a distal end, wherein the distal end of the pull wire is coupled with a distal end of the steerable section, and wherein the pull wire is configured to selectively control a curvature of the steerable section, and the pull wire is extending through the second lumen of the second tubular sleeve and is configured to pass through the steerable section to the distal end of the steerable section.

EXAMPLE 126: The delivery system of any examples herein, particularly examples 124-125, wherein the steerable section comprises: a slotted tube having a first straight position and a second curved position; and a flexible tubular portion and an exterior cover extending over at least a portion of the tubular portion, the cover being less flexible than the tubular portion.

EXAMPLE 127: The delivery system of any examples herein, particularly example 126, wherein the flexible tubular portion is a metal tubular portion comprising a plurality of axially spaced-apart, circumferentially extending openings, wherein each opening in the tubular portion of the steerable section comprises two elongate portions connected by a curved portion.

EXAMPLE 128: The delivery system of any examples herein, particularly example 127, wherein the second lumen of the second sleeve is circumferentially aligned with the curved portion of the openings of the steerable portion.

EXAMPLE 129: The delivery system of any examples herein, particularly examples 124-128, wherein the system further comprises a handle coupled to the proximal end of the elongated polymeric sleeve, wherein the handle is coupled to the proximal end of the pull wire.

EXAMPLE 130: The delivery system of any examples herein, particularly example 129, wherein the handle comprises a steering mechanism for actuating the pull wire to selectively control a curvature of the steerable section during advancement of the delivery, wherein the steering mechanism is effective to move the pull wire such that the elongated polymeric sleeve exhibits substantially no deflection during a movement of the pull wire.

EXAMPLE 131: A delivery system for deploying a prosthetic valve comprising: a first tubular sleeve having an inner polymer layer and an outer polymer layer, wherein the inner polymer layer defines an inner surface of the first tubular sleeve that forms a first lumen having a first diameter, and wherein the outer polymer layer is disposed on the inner polymer layer and defines an outer surface of the first tubular sleeve, and wherein the first tubular sleeve has a first longitudinal axis; and optionally, a second tubular sleeve that is substantially aligned with the first tubular sleeve, wherein the second tubular sleeve has an inner polymer layer and an outer polymer layer, wherein the inner polymer layer defines an inner surface of the second tubular sleeve that forms a second lumen having a second diameter, and wherein the outer polymer layer is disposed on the inner polymer layer and defines an outer surface of the second tubular sleeve, and wherein the second tubular sleeve has a second longitudinal axis; wherein the second diameter is substantially smaller than the first diameter; and wherein the second longitudinal axis is offset of the first longitudinal axis and is substantially parallel to the first longitudinal axis along a length of the elongated polymeric sleeve, wherein the inner polymer layer of the first tubular sleeve comprises a fluoropolymer; wherein the outer polymer layer of the first tubular sleeve comprises a polyamide, or a polyether block amide, or a combination thereof; and wherein the inner polymer layer and the outer polymer layers are coextruded.

EXAMPLE 132: The delivery system of any examples herein, particularly example 131, wherein the inner polymer layer is substantially free of delamination.

EXAMPLE 133: The delivery system of any examples herein, particularly example 131 or 132, wherein the fluoropolymer comprises an ethylene-perfluoroethylenepropene copolymer.

EXAMPLE 134: The delivery system of claims of any examples herein, particularly examples 131-133, wherein the outer polymer layer comprises polyamide comprising nylon 12.

EXAMPLE 135: The delivery system of any examples herein, particularly examples 131-134, wherein when the second tubular sleeve is present, the inner polymer layer and the outer polymer layer of the second tubular sleeve are the same as the inner polymer layer and the outer polymer layer of the first tubular sleeve.

EXAMPLE 136: The delivery system of any examples herein, particularly examples 131-135 further comprising an outer jacket, wherein the outer jacket is disposed outward of the outer surface of the outer polymer layer of the first tubular sleeve and outward of the outer surface of the second tubular sleeve if present, such that when the second tubular sleeve is present, the outer jacket encompasses both the first tubular sleeve and the second tubular sleeve.

EXAMPLE 137: The delivery system of any examples herein, particularly example 136, further comprising a braided layer disposed between the outer jacket and the outer surface of the outer polymer layer of the first tubular sleeve and the second tubular sleeve if present.

EXAMPLE 138: The delivery system of any examples herein, particularly example 137, wherein the inner and outer polymer layers of the first tubular sleeve and the second tubular sleeve if present and/or braided layer and/or the outer jacket are fused together such that when the second tubular sleeve is present the second tubular sleeve and the first tubular sleeve are inseparably coupled together to form an elongated polymeric sleeve.

EXAMPLE 139: The delivery system of any examples herein, particularly example 138, wherein the system further comprises a selectively steerable section having a proximal end and a distal end, wherein the proximal end of the steerable section is permanently coupled to the distal end of the elongated polymeric sleeve, wherein the steerable section comprises a central lumen having an inner diameter, wherein the central lumen is coaxial with the first lumen of the first tubular sleeve, and wherein the selectively steerable section is substantially more flexible than the elongated polymeric sleeve; and wherein the selectively steerable section is configured to provide a sufficient curvature to navigate around an aortic arch.

EXAMPLE 140: The delivery system of any examples herein, particularly example 139, further comprising a pull wire having a proximal end and a distal end, wherein the distal end of the pull wire is coupled with a distal end of the steerable section, and wherein the pull wire is configured to selectively control a curvature of the steerable section, and the pull wire is extending through the second lumen of the second tubular sleeve and is configured to pass through the steerable section to the distal end of the steerable section.

EXAMPLE 141: The delivery system of any examples herein, particularly examples 138-139, wherein the steerable section comprises: a slotted tube having a first straight position and a second curved position; and a flexible tubular portion and an exterior cover extending over at least a portion of the tubular portion, the cover being less flexible than the tubular portion.

EXAMPLE 142: The delivery system of any examples herein, particularly example 140, wherein the flexible tubular portion is a metal tubular portion comprising a plurality of axially spaced-apart, circumferentially extending openings, wherein each opening in the tubular portion of the steerable section comprises two elongate portions connected by a curved portion.

EXAMPLE 143: The delivery system of any examples herein, particularly examples 140-142, wherein the system further comprises a handle coupled to the proximal end of the elongated polymeric sleeve, wherein the handle is coupled to the proximal end of the pull wire.

EXAMPLE 144: The delivery system of any examples herein, particularly examples 142-143, wherein the handle comprises a steering mechanism for actuating the pull wire to selectively control a curvature of the steerable section during advancement of the delivery, wherein the steering mechanism is effective to move the pull wire such that the elongated polymeric sleeve exhibits substantially no deflection during a movement of the pull wire.

EXAMPLE 145: An assembly comprising: a self-expanding prosthetic valve comprising a radially compressible and expandable metallic stent and a flexible valvular structure mounted within the stent; and a delivery apparatus comprising a delivery system of any examples herein, particularly examples 116-144.

EXAMPLE 146: A method of making a delivery sleeve assembly comprising: forming an elongated polymeric sleeve having a proximal end and a distal end and comprising an inner polymer layer and an outer polymer layer; wherein the inner polymer layer forms: a first tubular sleeve having an inner surface defining a first lumen having a first diameter, an outer surface, and a first longitudinal axis; and a second tubular sleeve that is permanently adjacent to the first tubular sleeve, wherein the second tubular sleeve has an inner surface defining a second lumen having a second diameter, and an outer surface that is an extension of the outer surface of the first tubular sleeve and wherein the second tubular sleeve has a second longitudinal axis; wherein the second diameter is substantially smaller than the first diameter; and wherein the second longitudinal axis is offset of the first longitudinal axis and is substantially parallel to the first longitudinal axis along a length of the elongated polymeric sleeve; and wherein the outer surface of the first tubular sleeve and the second tubular sleeve defines an outer surface of the inner polymer layer; and wherein the inner surface of the first tubular sleeve defines an inner surface of the elongated polymeric sleeve, wherein the outer polymer layer extends circumferentially around the inner polymer layer, wherein the outer polymer layer has an inner surface and an outer surface, wherein the inner surface of the outer polymer layer is adjacent and bonded to the outer surface of the inner polymer layer; wherein the step of forming comprises co-extruding a first polymer to form the inner polymer layer and a second polymer to form the outer polymer layer.

EXAMPLE 147: The method of any examples herein, particularly example 146, wherein the first polymer comprises a fluoropolymer comprising an ethylene-perfluoroethylenepropene copolymer.

EXAMPLE 148: The method of any examples herein, particularly examples 146 or 147, wherein the second polymer comprises a polyamide, a polyether block amide, or a combination thereof.

EXAMPLE 149: The method of any examples herein, particularly example 148, wherein the polyamide comprises nylon 12.

EXAMPLE 150: The method of any examples herein, particularly examples 146-149, wherein the step of forming the elongated polymeric sleeve further comprises disposing an outer jacket on the outer surface of the outer polymer layer such that the inner polymer layer and the outer polymer layer are enclosed within the outer jacket.

EXAMPLE 151: The method of 150, any examples herein, particularly example further comprising a step of providing a braided layer over the outer surface of the outer polymer layer, prior to the step of disposing the outer jacket.

EXAMPLE 152: The method of any examples herein, particularly example 150 or 151, wherein the outer jacket comprises a polyether block amide.

EXAMPLE 153: The method of any examples herein, particularly examples 151-152, wherein the braided layer comprises a metal mesh or a laser cut hypo tube.

EXAMPLE 154: The method of any examples herein, particularly examples 151-154, wherein the method comprises a step of heat treatment to fuse the inner polymer layer, the outer polymer layer, the braided layer, and the outer jacket together.

EXAMPLE 155: A method of making a delivery sleeve assembly comprising: forming a first tubular sleeve having an inner polymer layer and an outer polymer layer, wherein the inner polymer layer defines an inner surface of the first tubular sleeve that forms a first lumen having a first diameter, and wherein the outer polymer layer is disposed on the inner polymer layer and defines an outer surface of the first tubular sleeve, and wherein the first tubular sleeve has a first longitudinal axis; and optionally, forming a second tubular sleeve that is substantially aligned with the first tubular sleeve, wherein the second tubular sleeve has an inner polymer layer and an outer polymer layer, wherein the inner polymer layer defines an inner surface of the second tubular sleeve that forms a second lumen having a second diameter, and wherein the outer polymer layer is disposed on the inner polymer layer and defines an outer surface of the second tubular sleeve, and wherein the second tubular sleeve has a second longitudinal axis; wherein the second diameter is substantially smaller than the first diameter; and wherein the second longitudinal axis is offset of the first longitudinal axis and is substantially parallel to the first longitudinal axis along a length of the elongated polymeric sleeve, wherein the inner polymer layer of the first tubular sleeve comprises a fluoropolymer; wherein the outer polymer layer of the first tubular sleeve comprises a polyamide, or a polyether block amide, or a combination thereof; and wherein the step of forming comprises co-extruding a first polymer to form the inner polymer layer of the first tubular sleeve and optionally of the second tubular sleeve and a second polymer to form the outer polymer layer of the first tubular sleeve and optionally of the second tubular sleeve.

EXAMPLE 156: The method of any examples herein, particularly example 155, wherein the inner polymer layer of the first tubular sleeve or the second tubular sleeve, if present, is substantially free of delamination.

EXAMPLE 157: The method of any examples herein, particularly example 155 or 156, wherein the first polymer comprises a fluoropolymer comprising an ethylene-perfluoroethylenepropene copolymer.

EXAMPLE 158: The method of any examples herein, particularly examples 155-157, wherein the polyamide comprises nylon 12.

EXAMPLE 159: The method of any examples herein, particularly examples 155-158, wherein when the second tubular sleeve is present, the inner polymer layer and the outer polymer layer of the second tubular sleeve are the same as the inner polymer layer and the outer polymer layer of the first tubular sleeve.

EXAMPLE 160: The method of any examples herein, particularly examples 155-159 further comprising disposing an outer jacket, outward of the outer surface of the outer polymer layer of the first tubular sleeve and outward of the outer surface of the second tubular sleeve if present, such that when the second tubular sleeve is present, the outer jacket encompasses both the first tubular sleeve and the second tubular sleeve.

EXAMPLE 161: The method of any examples herein, particularly example 160, further comprising disposing a braided layer between the outer jacket and the outer surface of the outer polymer layer of the first tubular sleeve and the second tubular sleeve if present prior to the step of disposing the outer jacket.

EXAMPLE 162: The method of any examples herein, particularly example 161, further comprising fusing the inner and outer polymer layers of the first tubular sleeve and the second tubular sleeve if present and/or braided layer and/or the outer jacket such that when the second tubular sleeve is present the second tubular sleeve and the first tubular sleeve are inseparably coupled together to form an elongated polymeric sleeve.

EXAMPLE 163: The method of any examples herein, particularly examples 160-162, wherein the outer jacket comprises a polyether block amide.

EXAMPLE 164: The method of any examples herein, particularly examples 160-163, wherein the braided layer comprises a metal mesh or a laser cut hypo tube.

Although several aspects of the disclosure have been disclosed in the foregoing specification, it is understood by those skilled in the art that many modifications and other aspects of the disclosure will come to mind to which the disclosure pertains, having the benefit of the teaching presented in the foregoing description and associated drawings. It is thus understood that the disclosure is not limited to the specific aspects disclosed hereinabove and that many modifications and other aspects are intended to be included within the scope of the appended claims. Moreover, although specific terms are employed herein, as well as in the claims which follow, they are used only in a generic and descriptive sense and not for the purposes of limiting the described disclosure nor the claims which follow. We, therefore, claim as our disclosure all that comes within the scope and spirit of these claims.

1. A delivery system for deploying a prosthetic valve comprising:
an elongated polymeric sleeve having a proximal end and a distal end and comprising an inner polymer layer and an outer polymer layer;
wherein the inner polymer layer forms:
a first tubular sleeve having an inner surface defining a first lumen having a first diameter, an outer surface, and a first longitudinal axis; and
a second tubular sleeve that is adjacent to the first tubular sleeve, wherein the second tubular sleeve has an inner surface defining a second lumen having a second diameter, and an outer surface that is an extension of the outer surface of the first tubular sleeve and wherein the second tubular sleeve has a second longitudinal axis;
wherein the second diameter is substantially smaller than the first diameter; and wherein the second longitudinal axis is offset of the first longitudinal axis and is substantially parallel to the first longitudinal axis along a length of the elongated polymeric sleeve; and
wherein the outer surface of the first tubular sleeve and the second tubular sleeve defines an outer surface of the inner polymer layer; and
wherein the inner surface of the first tubular sleeve defines an inner surface of the elongated polymeric sleeve,
wherein the outer polymer layer extends circumferentially around the inner polymer layer, wherein the outer polymer layer has an inner surface and an outer surface, wherein the inner surface of the outer polymer layer is adjacent and bonded to the outer surface of the inner polymer layer;
wherein the inner polymer layer comprises a fluoropolymer; and
wherein the outer polymer layer comprises a polyamide, or a polyether block amide, or a combination thereof.

2. The delivery system of claim 1, wherein the inner polymer layer and the outer polymer layer of the elongated polymeric sleeve are co-extruded.

3. The delivery system of claim 1, wherein the fluoropolymer comprises an ethylene-perfluoroethylenepropene copolymer, wherein the outer polymer layer is the polyamide comprising nylon 12.

4. The delivery system of claim 1, wherein the elongated polymeric sleeve further comprises an outer jacket, wherein the outer jacket is disposed outward of the outer surface of the outer polymer layer of the elongated polymeric sleeve,
wherein the elongated polymeric sleeve further comprises a braided layer disposed between the outer jacket and the outer surface of the outer polymer layer of the elongated polymeric sleeve,
wherein the inner and outer polymer layers of the elongated polymeric sleeve and/or the braided layer and/or the outer jacket are fused together,
wherein the outer jacket comprises a polyether block amide.

5. The delivery system of claim 1 further comprising:
a selectively steerable section having a proximal end and a distal end, wherein the proximal end of the steerable section is coupled to the distal end of the elongated polymeric sleeve,
wherein the steerable section comprises a central lumen having an inner diameter,
wherein the central lumen is coaxial with the first lumen of the first tubular sleeve,
wherein the selectively steerable section is substantially more flexible than the elongated polymeric sleeve, and
wherein the selectively steerable section provides a sufficient curvature to navigate around an aortic arch.

6. The delivery system of claim 5 further comprising:
a pull wire having a proximal end and a distal end, wherein the distal end of the pull wire is coupled with a distal end of the steerable section, and wherein actuation of the pull wire selectively controls a curvature of the steerable section, and the pull wire is extending through the second lumen of the second tubular sleeve and is configured to pass through the steerable section to the distal end of the steerable section.

7. The delivery system of claim 5, wherein the steerable section comprises:
a slotted tube having a first straight position and a second curved position; and
a flexible tubular portion and an exterior cover extending over at least a portion of the tubular portion, the exterior cover being less flexible than the tubular portion,
wherein the flexible tubular portion is a metal tubular portion comprising a plurality of axially spaced-apart, circumferentially extending openings, wherein each of the openings in the tubular portion of the steerable section comprises two elongate portions connected by a curved portion,
wherein the second lumen of the second tubular sleeve is circumferentially aligned with the curved portion of the openings of the steerable section.

8. The delivery system of claim 6 further comprising:
a handle coupled to the proximal end of the elongated polymeric sleeve, wherein the handle is coupled to the proximal end of the pull wire,
wherein the handle comprises a steering mechanism that actuates actuating the pull wire to selectively control a curvature of the steerable section during advancement of the delivery, wherein the steering mechanism is effective to move the pull wire such that the elongated polymeric sleeve exhibits substantially no deflection during a movement of the pull wire.

9. A delivery system for deploying a prosthetic valve comprising:
a first tubular sleeve having an inner polymer layer and an outer polymer layer, wherein the inner polymer layer defines an inner surface of the first tubular sleeve that forms a first lumen having a first diameter, and wherein the outer polymer layer is disposed on the inner polymer layer and defines an outer surface of the first tubular sleeve, and wherein the first tubular sleeve has a first longitudinal axis; and
a second tubular sleeve that is substantially aligned with the first tubular sleeve, wherein the second tubular sleeve has an inner polymer layer and an outer polymer layer, wherein the inner polymer layer defines an inner surface of the second tubular sleeve that forms a second lumen having a second diameter, and wherein the outer polymer layer is disposed on the inner polymer layer and defines an outer surface of the second tubular sleeve, and wherein the second tubular sleeve has a second longitudinal axis; wherein the second diameter is substantially smaller than the first diameter; and wherein the second longitudinal axis is offset of the first longitudinal axis and is substantially parallel to the first longitudinal axis along a length of the elongated polymeric sleeve,
wherein the inner polymer layer of the first tubular sleeve comprises a fluoropolymer; wherein the outer polymer layer of the first tubular sleeve comprises a polyamide, or a polyether block amide, or a combination thereof; and
wherein the inner polymer layer and the outer polymer layers are coextruded.

10. The delivery system of claim 9, wherein the inner polymer layer is substantially free of delamination,
wherein the fluoropolymer comprises an ethylene-perfluoroethylenepropene copolymer,
wherein the outer polymer layer comprises polyamide comprising nylon.

11. The delivery system of claim 9, wherein the inner polymer layer and the outer polymer layer of the second tubular sleeve correspond to the inner polymer layer and the outer polymer layer of the first tubular sleeve, respectively.

12. The delivery system of claim 9 further comprising:
an outer jacket, wherein the outer jacket is disposed outward of the outer surface of the outer polymer layer of the first tubular sleeve and outward of the outer surface of the second tubular sleeve if present, such that when the second tubular sleeve is present, the outer jacket encompasses both the first tubular sleeve and the second tubular sleeve.

13. The delivery system of claim 12 further comprising:
a braided layer disposed between the outer jacket and the outer surface of the outer polymer layer of the first tubular sleeve and the second tubular sleeve if present,
wherein the inner and outer polymer layers of the first tubular sleeve and the second tubular sleeve if present and/or the braided layer and/or the outer jacket are fused together such that when the second tubular sleeve is present, the second tubular sleeve and the first tubular sleeve are inseparably coupled together to form an elongated polymeric sleeve.

14. The delivery system of claim 9 further comprising:
a selectively steerable section having a proximal end and a distal end, wherein the proximal end of the steerable section is coupled to the distal end of the elongated polymeric sleeve,
wherein the steerable section comprises a central lumen having an inner diameter,
wherein the central lumen is coaxial with the first lumen of the first tubular sleeve,
wherein the selectively steerable section is substantially more flexible than the elongated polymeric sleeve,
wherein the selectively steerable section provides a sufficient curvature to navigate around an aortic arch.

15. The delivery system of claim 14 further comprising:
a pull wire having a proximal end and a distal end, wherein the distal end of the pull wire is coupled with a distal end of the steerable section, and wherein actuation of the pull wire selectively controls a curvature of the steerable section, and the pull wire is extending through the second lumen of the second tubular sleeve and is configured to pass through the steerable section to the distal end of the steerable section.

16. The delivery system of claim 14, wherein the steerable section comprises:
a slotted tube having a first straight position and a second curved position; and
a flexible tubular portion and an exterior cover extending over at least a portion of the tubular portion, the exterior cover being less flexible than the tubular portion.

17. The delivery system of claim 16, wherein the flexible tubular portion is a metal tubular portion comprising a plurality of axially spaced-apart, circumferentially extending openings, wherein each opening in the tubular portion of the steerable section comprises two elongate portions connected by a curved portion.

18. The delivery system of claim 15 further comprising:
a handle coupled to the proximal end of the elongated polymeric sleeve, wherein the handle is coupled to the proximal end of the pull wire, wherein the handle comprises a steering mechanism that actuates the pull wire to selectively control a curvature of the steerable section during advancement of the delivery, wherein the steering mechanism is effective to move the pull wire such that the elongated polymeric sleeve exhibits substantially no deflection during a movement of the pull wire.

19. A method of making a delivery sleeve assembly comprising:
forming an elongated polymeric sleeve having a proximal end and a distal end and comprising an inner polymer layer and an outer polymer layer;
wherein the inner polymer layer forms:
a first tubular sleeve having an inner surface defining a first lumen having a first diameter, an outer surface, and a first longitudinal axis; and
a second tubular sleeve that is adjacent to the first tubular sleeve, wherein the second tubular sleeve has an inner surface defining a second lumen having a second diameter, and an outer surface that is an extension of the outer surface of the first tubular sleeve and wherein the second tubular sleeve has a second longitudinal axis;
wherein the second diameter is substantially smaller than the first diameter; and wherein the second longitudinal axis is offset of the first longitudinal axis and is substantially parallel to the first longitudinal axis along a length of the elongated polymeric sleeve; and
wherein the outer surface of the first tubular sleeve and the second tubular sleeve defines an outer surface of the inner polymer layer; and
wherein the inner surface of the first tubular sleeve defines an inner surface of the elongated polymeric sleeve,
wherein the outer polymer layer extends circumferentially around the inner polymer layer, wherein the outer polymer layer has an inner surface and an outer surface, wherein the inner surface of the outer polymer layer is adjacent and bonded to the outer surface of the inner polymer layer; and
wherein the step of forming comprises co-extruding a first polymer to form the inner polymer layer and a second polymer to form the outer polymer layer.

20. The method of claim 19, wherein the step of forming the elongated polymeric sleeve further comprises disposing an outer jacket on the outer surface of the outer polymer layer such that the inner polymer layer and the outer polymer layer are enclosed within the outer jacket,
the method further comprises:
providing a braided layer over the outer surface of the outer polymer layer prior to the step of disposing the outer jacket,
applying a heat treatment to fuse the inner polymer layer, the outer polymer layer, the braided layer, and the outer jacket together.
